SEQUENCE LISTING (9 sequences)

SEQ ID NO: 1 - protein, 908 aa, Arabidopsis thaliana
MGSNRPKNFRRRGDDGGDEIDGKVATPSSKPTSTLSSSKPKTLSASAPKKKLLSFADDEEEEEDGAPRVTIKPKNGRDRVKSSSRLGVSGSSHRHSSTKERRPASSNVLPQAGSYSKEALLELQKNTRTLPYSRSSANAEPKVVLKGLIKPPQDHEQQSLKDVVKQVSDLDFDEEGEEEQHEDAFADQAAIIRAKKERMRQSRSAPAPDYISLDGGIVNHSAVEGVSDEDADFQGIFVGPRPQKDDKKGVFDFGDENPTAKETTTSSIYEDEDEEDKLWEEEQFKKGIGKRMDEGSHRTVTSNGIGVPLHSKQQTLPQQQPQMYAYHAGTPMPNVSVAPTIGPATSVDTLPMSQQAELAKKALKDNVKKLKESHAKTLSSLTKTDENLTASLMSITALESSLSAAGDKYVFMQKLRDFISVICDFMQNKGSLIEEIEDQMKELNEKHALSILERRIADNNDEMIELGAAVKAAMTVLNKHGSSSSVIAAATGAALAASTSIRQQMNQPVKLDEFGRDENLQKRREVEQRAAARQKRRARFENKRASAMEVDGPSLKIEGESSTDESDTETSAYKETRDSLLQCADKVFSDASEEYSQLSKVKARFERWKRDYSSTYRDAYMSLTVPSIFSPYVRLELLKWDPLHQDVDFFDMKWHGLLFDYGKPEDGDDFAPDDTDANLVPELVEKVAIPILHHQIVRCWDILSTRETRNAVAATSLVTNYVSASSEALAELFAAIRARLVEAIAAISVPTWDPLVLKAVPNTPQVAAYRFGTSVRLMRNICMWKDILALPVLENLALSDLLFGKVLPHVRSIASNIHDAVTRTERIVASLSGVWTGPSVTRTHSRPLQPLVDCTLTLRRILEKRLGSGLDDAETTGLARRLKRILVELHEHDHAREIVRTFNLKEAV

SEQ ID NO: 2 - protein, 913 aa, Glycine max
MSTAKSRNFRRRGGDTESNDGNDGGTTTTTFPSKPTSSAKPKKKPQAPKLLSFADEDEQTDENPRPRASKPYRSAATAKKPSSSHKITTLKDRIAHSSSPSVPSNVQPQAGTYTKEALRELQKNTRTLVTSSSSRSDPKPSSEPVIVLKGLVKPLGSEPQGRDSYSEGEHREVEAKLATVGIQNKEGSFYPDDETIRAIRAKRERLRQARPAAPDYISLDGGSNHGAAEGLSDEEPEFRGRIAMFGEKVDGGKKGVFEEVEERIMDVRFKGGEDEVVDDDDDDEEKMWEEEQFRKGLGKRMDEGSARVDVSVMQGSQSPHNFVVPSAAKVYGAVPSAAASVSPSIGGVIESLPALDVVPISQQAEAARKALLENVRRLKESHGRTMSSLSKTDENLSASLLNITALENSLVVADEKYRFMQKLRNYVTNICDFLQHKAFYIEELEEQMKKLHEDRALAISERRATNNDDEMIEVEEAVKAAMSVLSKKGNNMEAAKIAAQEAFSAVRKQRDLPVKLDEFGRDLNLEKRMNMKAKTRSEACQRKRSQAFDSNKVTSMELDDHKIEGESSTDESDSESQAYQSQSDLVLQAADEIFSDASEEYGQLSLVKSRMEEWKREHSSSYKDAYMSLSLPLIFSPYVRLELLRWDPLHNGVDFQEMKWYKLLFTYGLPEDGKDFVHDDGDADLELVPNLVEKVALPILHYEISHCWDMVSQQETVNAIAATKLMVQHVSHESEALADLLVSIQTRLADAVADLTVPTWSPSVLAAVPDAARVAAYRFGVSVRLLRNICLWKDVFSMPVLEKVALDELLCRKVLPHLRVISENVQDAITRTERIIASLSGIWAGPSVIGDKNRKLQPLVTYVLSLGRILERRNVPENDTSHLARRLKKILADLNEYDHARNMARTFHLKEAL

SEQ ID NO: 3 - DNA, 2839 bp, Glycine max
  source 1..2839: mol_type = other DNA
aggcttaggatccaagataatcaccaaaacacttcactccaaccatcacatccggtcaccggaacccaaacaatggcttccaccgccaaatcccgcaacttccgccgccgcggcggcgacgacaccgaatccaacgacgacaacgacggcgacaccacctcaaccaccctcccttccaagcccccttcctccgccaagcccaagaagaagccccaagcccccaagctcctcagcttcgccgatgacgaagacgaaaccgacgaaaacccgcgtccacgcgcctccaaaccccaccgtaccgccgccaccgccaaaaaaccctcgtcctctcacaaaatcaccactctcaaggaccgaatcgcgcatacctcttccccttcggtccctaccaacgtccaaccgcaagccggaacttacaccaaagaagccctacgtgaactccagaagaacacgcgaacgttggtctctagcagctcctctcgctccgatcccaaaccctcctccgagcctgtcatcgtattgaagggacacgtgaaacccttaggtccggagacccagggtcgggactccgattccgattccgaaggcgagcatagagaagttgaagccaaattggccaccgtgggcattcagaacaaggaggactcgttttatcctgatgaggaaaccatcagagctatccgggcgaagcgggagcggctccggctggcgcgccccgccgcgccggattacatctccttggatggtgggagcaatcatggcgctgctgaggggctgagcgacgaggagccagagtttaggggccggattgcgatgtttggggagaaggtggatggtgggaagaagggtgtttttgaggaggtggaggagaggagagtggatttgaggttcaagggtggagaggaagaggtgctggatgatgacgacgatgaggaggagaagatgtgggaagaggagcagtttaggaaggggttagggaagagaatggatgaagggtctgctagggttgatgttgctgctgctgctgtgcaaggtgctcaattgcagcacaattttgttgttccctctgcagcaaaggtgtatggtgcagttccaagtgcagcagcatcggtgagtcccagcatcggaggagcaattgaatcgttgccggttttggatgtcgtgcctatatcgcaacaagccgaggctgctaggaaggctttgcttgagaatgtgaggaggcttaaggaatctcatggaagaactatgtcatcattgagtaaaactgatgaaaatttatctgcctcccttttgaacatcactgctcttgaaaactctttagtagtagctgatgagaagtaccggtttatgcaaaagctccgaaattatgtcacaaatatatgtgacttcttacaacataaggcctgctacattgaggaacttgaagagcagatgaagaaacttcatcaagatcgtgcatcagccatttttgaaagaagagccactaacaatgatgatgaaatggttgaggtagaagaagctgtaaaagctgcaatgtcagttttaatcaagaaaggtaacaatatggaagccgccaaaattgctgctcaggaagcatttgctgctgtaaggaagcaaagggatctgccagtgaagttagatgaatttggtagagatttaaatcttgagaaacgaatgaatatgaaagtgagggccgaggcttgtcaacgcaagaggtcactagcatttggttataataaggtcacatccatggaatgggatgatcataaaatagaaggtgaatcaagcactgatgagagtgatagtgagagccaagcatatcagtcacaaagtgatttggtgctgcaagcagctgatgagattttcagtgatgcttctgaggaatatggtcaactatccttggtcaaaagtagaatggaagaatggaaaagagagtactcatcaacctataaagatgcttatatgtcattgagtcttccactgatcttctctccgtatgtaagattggaactcttgaggtgggacccactccacaagggtgtagattttcaagagatgaaatggtataaattgttgttcacttatggtttgcctgaagatggaaaagattttgttcatgatgatggagatgctgaccttgaacttgtcccaaacttggtggaaaaggttgcacttcctattctgcactatgaaatttcccattgctgggacatgcttagtcaacaagaaacagtgaatgctattgctgctacaaaattgattgtgcagcatgtgtctcatgaaagtgaagctcttgctggtttgctagtttccattcgaacacgcttagctgatgctgttgctaatcttacagtcccaacatggagtctgcctgtactagctgctgttccagatgctgcacgagttgcggcgtatcggtttggtgtgtctgttcgattgttgagaaatattggctcgtggaaggatgtattttcaatggcggtattagagaaggttgctcttgatgaacttttgtgcggaaaagttctacctcacttaagagtcatatcagaaaatgttcaagatgcaataacaagaacagaacggattattgcttccctatctggggtctggtctggtccaagtgtcattggagacaaaaaccgcaaattgcagcctctggtgacttatgtgctgtcacttggaaggattcttgagagaagaaatgtgccagagagtgacacaagccatctagctcgtcgattaaagaaaatacttgttgacctcaatgaatatgatcatgctaggagcatggctagaaccttccatctcaaggaggcattgtgatagactagtaacc

SEQ ID NO: 4 - DNA, 3728 bp, Glycine max
  source 1..3728: mol_type = other DNA
tattttaaaaagaattatttaaaagttgtatttttttaaaagttgtatcatctatgtggttatttaaaggtgaaagtcttcatgctgaagggattgctattgtatatttttttccgcaactgttgtccaaaatgtttgacacgcgaaaggtgttttttagttttggcaaataaactcttgcagagctcaagcattagcacaggaaaacatcattcttggccatgtggatgtaatggaagatgaatgttcattgtcttaatattagattatcccctggtgggctggttagccataaatttctttagagtacacagatgtcaaaataataaccatacttttagaatcgtgtgcttttttatgtttgaaaggttggagttggagcaatcctgatcccaattgcatgctggacaataaataaaaagtacctaaaattaaggacatattcataattgatgatcaaatgtcaagaatttgaaaactaaacacacgtcaataacaacaaatgttgaagaatcaaacaaagttcaagcctctaagactaaagtcaataacaacaaatatcaaagaatcaaataagtttaagcctctaggacttgaagcaactaaatatcagattcaaaataaaagaattttcttaatagtttctactttctagaatgggatttctgattggattaaatgatgaaggatgtgaaatcaagaaggattttcactcatgtacagaatagtggctgagatggctgcaatttcatgcaaaagtggccataattgtactgctacgaccgtgacacaaaactgctccacagcaggaggccacaatccaattaaactatagtggcactatcatttgggttgactactatgctataaaccacttgaaaaacatgacggtgatacttctaataatgttgaagaatatgtattttctttttgctggatacttaaaagttgatactatattatctgcttatcatttgtgaatgcttttatgcatgtcattatttattgaggctgtaactaaagttatcctatgacttgtacttcagcataaagcttttttacattgaggaacttgaagagcagatgaagaaacttcatgaagatcgtgcatcagccatttttgaaagaagaaccactaacaatgatgatgaaatgattgaggtggaagcagctataaaagctgcaatgtcagttttagacaagaaaggtaacaatatggaggctgccaaatctgctgctcaggaagcatttgctgctgtaagaaaacaaaaagatctgccagtgaagttagatgaatttggtagagatttaaatattgagaaacagatgcaaatgaaagtgagggctgaggctcgtcaacgcaagaggtcacaagcatttaattctaataagctggcatacatggaactggatgatcctaaaatagagggtgaatcaaacactgatgagagtgatagtgagagtcaagcatatcagtcacagcgtgatttggtgcagcgggctgctgatgagattttcagtgaagcttctgaggaatatggccaattatcctttgtcaaaagaagaatggaagaatggaaaagagagtattcatcaagctataaagatgcttatatgtcattgaatcttccactggttttctctccatatgtaagattggaacttctgaggtgggacccacttcacaagggtctagattttcaagagatgaaatggtataaattattgttcacttatggtttgcctgaagatggaaaagattttgttcaagatgatggagatgctgaccttgaacttgtcccgaacttggtggcaaaggttgcacttcctattctgcactacgaaatttcccattgctgggacatgcttggtcaacaggaaacagtgaatgctattgctgctacaaaattgattgtgcaacaagtatctcatgaaagtgaagctcttgctgattataattttcttattttacacccacaatgaatttactcgtaggtgttggttcattttctctttttggttacttcgagttctttcttgaataaaattagaggtatttgatagttaatatttagcttgtttttcaaattctcctattttttatatgtgggacaccttcaacataaatctttgggaaggtgatgatcttcaaaacttcaaatatgatattcttccctgctgatcaatcaatccattgccattgggataagacatgattgtagcaaataagccgaattttcgttggttttgaagcagaaacactacattggaactgctcagaaactgactataaatgtataaggaatgtgctatatgtgataaaacattttgcttctgttgtgagatatcagactttaaattttgatacttaatgctaaaatactatggttgttgtatcacattaccagctgagatttccctttgataacaaatttttaatgcattattttttattttggttgttgttgaatagagagttggctataagcaagagtacataggtgagtgagacattggccatgagcccaggattagaggtcaattctcagcattggaagattaatcagatgtgggccgtgggggacatgagactaactaagaattgggcattgtgagtaaatatttttgaggaagaacatgtagtgtgaaactatgaatctagtaaggaaatttaggttgtgaatgaacagaggaatagaaacgctttgtaacgtgggtatatgtagataccatattgtgagattgtgtttcttgagttgtgagagatgcaccaccttatgaaaatgctgttaatttgattaactaagttagaagtagagtgtcacagtaagccataatgagttaatagtatgtaattatctcaatgcatacttaaagttggatttggtactgacatgtgttcatttttctttactggaaattttgagcttcattgaatatgggcaaaatgtgttggcttgttaatgacttatgattttttataattcttcctgctttaggcaatgctcagaattgatttatgctcttgctgatgaaagaaatgtttccgattgattatactacaaatgttgaagaaaacatttcattgcttatgttttatcttaacacctttttcattgttgattaatcaccgtcaaccagtattgaagggggaaccaaatggtttgtatctactatctataacatttcaatgtcttgctcaaacatttgtacaaatatggtggtagtctagtgtaaacagggacattgcactgcttttccagaaactacttttgctgaaaaacaattagataaactgagaaggactccaaaacattctgaactggaattaatgtaaaaaacatgcattgtaagatatgcattgtcatgtatgaccaaatcatcttgtacagaaatgttttggatttttacgtttgatctttagtgcatacaaattacatatttgaatttcaccaaaaaaaaataataattacatatttgaatagcatcccacttttatgaatcttaaaaatctgttaagtccctataaagatgatttatacattcttcatacagggaacgaagacaaggtactttatgggtaaaactgaccgtggtttcatctggggtatcaaagctccatttcacagctggcatgaagaaaactaggaaaatggaagaagcatataacgtttctatagatggtataaccgtggataaattctaatattaacatccttttgttaaaa

SEQ ID NO: 5 - DNA, 4725 bp, Glycine max
  source 1..4725: mol_type = other DNA
cagtatcgggtaattttccctccctcgcaatctgttcaatacttctttcacacttggaatctgaaatcacaaaacccttcactccaaccatcacctccggtcaccggaaccctcccccatgtccaccgccaaatcccgcaacttccgccgccgtggcggcgacaccgaatctaatgacggcaacgacggcggcaccaccaccaccacttttccttccaagcccacttcctccgccaagcccaagaagaagcctcaagcgccgaagctcctcagcttcgccgatgaagacgaacaaactgacgaaaacccgcgtccacgcgcctccaaaccctaccgttccgccgccaccgccaaaaaaccgtcgtcctctcacaaaatcaccactctcaaggaccgaatcgcgcattcctcttccccttcagtcccttccaacgttcagccccaagccggaacgtacaccaaagaagccctacgcgagctccagaagaacacgcgaacgttggtcactagcagctcctctcgctccgatcccaaaccctcctccgaacctgtcattgtcctgaagggactcgtgaaacccttaggctcggagccccagggtcgggattcctattccgaaggggaacatagagaagttgaagcaaaattggccactgtgggcattcagaataaggagggctcgttttatcctgatgatgaaaccatcagggctatccgggcgaagcgggagcgcctccggcaggcgcgccctgctgcgccggattatatctcgttggatggtgggagcaatcatggtgctgctgaggggctgagcgacgaggagccggagtttcgggggcggattgcgatgtttggggagaaggtggatggtgggaagaagggtgtttttgaggaggtggaggagaggataatggatgtgaggttcaagggtggagaggatgaggtggtggatgatgatgatgatgatgaggagaagatgtgggaagaggagcagtttaggaaagggttagggaagagaatggatgaagggtctgctagggttgatgtttctgttatgcaaggttctcaatcgccgcacaattttgttgttccctctgcagcaaaggtgtatggtgcagttccaagtgctgcagcatcagtgagtcccagcattggaggagtgattgaatcgttgccggctttggatgttgtgcccatatcgcaacaagctgaggctgctaggaaagctctgctagagaatgtgaggaggcttaaggaatctcatggaagaacaatgtcatcattgagtaaaactgatgaaaatttgtctgcctcgcttttgaacatcactgctcttgaaaactctttagtggtagctgatgagaagtaccggtttatgcaaaagctccgaaattatgtcacaaatatatgtgactttttacagcataaagcattttacattgaggaacttgaagagcagatgaagaaacttcatgaagatcgtgcattagccatttctgaaagaagagccactaacaatgatgatgaaatgattgaggtagaagaagctgtaaaagctgcaatgtcagttttaagcaagaaaggtaacaatatggaagctgccaaaattgctgctcaggaagcattttctgctgtaagaaaacaaagagatctgccagtgaagttagatgaatttggtagagatctaaatcttgagaaacgaatgaatatgaaggcaaaaacaaggtctgaggcttgtcaacgtaagaggtcacaagcatttgattctaataaggtcacatccatggaattggatgatcataaaatagaaggtgaatcaagcactgatgagagtgatagtgagagtcaagcatatcagtcacaaagtgatttggtgctgcaggctgctgatgagattttcagtgatgcttctgaggaatatggtcaattatccttggtcaaaagtagaatggaagaatggaaaagagagcattcgtcaagctataaagatgcttatatgtcattgagtcttccactgatcttctctccatatgtaagattggaactcctgaggtgggacccacttcacaatggtgtagattttcaagagatgaaatggtataaattgttgttcacttatggtttgcctgaagatggaaaagattttgttcacgatgatggagatgctgacctcgaacttgtcccaaacttggtggaaaaggttgcacttcctattctgcactatgaaatttcccattgctgggacatggtgagtcaacaagaaacagtgaatgctattgctgctacaaaattgatggtgcagcatgtgtctcatgaaagtgaagctcttgctgatttgctagtttccattcaaacacgcttagctgatgctgttgctgatcttacagtcccaacatggagtccgtctgtactagctgctgttccagatgctgcacgagttgcagcatatcggtttggtgtgtctgttcgattgttgagaaatatttgcttgtggaaggatgtattttcaatgccagtattagagaaggttgctctagatgagcttttgtgcagaaaagttctacctcacttaagagtcatatcagaaaatgttcaagatgcaataacaagaactgaacggattattgcttccctatctgggatctgggctggtccaagtgtcattggagacaaaaaccgtaaattgcagcctctggtgacttatgtgctgtcacttggaaggattctggagagaagaaatgtgccagaaaatgacacaagtcatctagctcgtcgattaaagaaaatactagctgacctcaatgaatatgatcatgctaggaacatggctagaaccttccatctcaaggaggcattgtgagcttgcttggaattcatgttttgttataattggtgtcttggtttgttgtggagtctggccctaggttaacccttcttggtgcaattttagatagctttttgacctttacacaagtcttcatcagagtatgcttatgattgcatcttgaccttcaaatcctgaggaggcatcattctctcttacaatagagggatgaagacttaatggaacatttgtacctaattgaggacctccattgctccttgtgaaccacgattaggaaccaaataatcattattccctatctctatttgggggtgcagagggagtttcattttatcttttttcattacctatgcttatgctagagaccctggaggacattgtaaaattttccatactcatctaattatagatcacatttgatattgatatgaaataacttgtataaggtccattctaacattatttttggtaattgagtggatttggtggcagaggtatttatattatggttttaaattctaataaatgatgtgaagatatactttctctccttttcatttttcctgccaaattgtcaccaagtcagtgtgaatggtgtttccaaaatgtttacagaatgcaagttttagatcagacattaagagatcctaatgacctctaacccttttttcatgtgcttagtcttaggtcttatgtggaaatgtgcaggatatgcagaatgggagattcttgccctatagacctcatccctaggccttaagtctgtaggctaagtctgaaaggcccatagttgttaatagcgggcgtcgcggcacggcgcccatccgcgacgaagaggaacgtagcggcgcgggcgtcgtggccgctatcgcggcggagcggcgcgggatctggaccggattttcgcaccgctacagtgacgccgtgaaaattttgaaaatgccctcacttaatgcgggaaagtgagagtatttttggtattttgctttttgtattactcggccaactctccgtccagaccgctcaaaaaccctagtctccactctccagacctgcaactgcgaccggcgaagctgcgaggacgacgacgagtctgcatcctgcgaagctgcgaccctgacacctgcgtcctgcgtccggcgaccccgacgacgagtctgcgctgcgccgccaccttgcttgaggtgaggaagacgtgcgatgagctgttgacccctatagcattttttttgtgtgtgacactgtgtgactgtgatttttttatttgttttttgaaaaatgttatatgattttttgtgtgtgtgactgtgactatgaaaagaatgcttattaaattgttttgtgcgtcctgcgtaggctttagtgattttttttaattaaattgtttgaggcctttcacagaagaaatctatctaaatatgcttattaatttttgtttctagagaaaatttaattgctccccatataagataggtggttatgtactgtaactcaatattcatctggtggatgttacttgtgtgttcagttttggcaatgtcattttgatatttggtctcgaccaggaacgatccatataaaccaattatcccagcattcatttcactttttgggctattttttaagtattcggcccaatctttcagtggtatgaaatcagatgttgcaaaattcatttctaataaaaatggttatgaaaaggcttgatacaatagttccaattattcctctaattagatcattggcgaaagcaaaattttgtaatgtatttggccatcccattagtaagccggtttgggccaatttatctgattttgatattattgaccagtttttgcggatatgccaaaatttttctcattattacaatggatatgccg

SEQ ID NO: 6 - protein, 575 aa, Arabidopsis thaliana
MATHSMIIPSPSSSSSSLATAASPFKETLPLFSRSLTFPRKSLFSQVRLRFFAEKHSQLDTSNGCATNFASLQDSGDQLTEEHVTKGESTLSITVVGASGDLAKKKIFPALFALFYEGCLPQDFSVFGYARTKLTHEELRDMISSTLTCRIDQREKCGDKMEQFLKRCFYHSGQYNSEEDFAELNKKLKEKEAGKISNRLYYLSIPPNIFVDVVRCASLRASSENGWTRVIVEKPFGRDSESSGELTRCLKQYLTEEQIFRIDHYLGKELVENLSVLRFSNLVFEPLWSRNYIRNVQLIFSEDFGTEGRGGYFDQYGIIRDIMQNHLLQILALFAMETPVSLDAEDIRSEKVKVLRSMKPLRLEDVVVGQYKGHNKGGKTYPGYTDDPTVNHSLTPTFAAAAMFINNARWDGVPFLMKAGKALHTRGAEIRVQFRHVPGNLYKKSFATNLDNATNELVIRVQPDEGIYLRINNKVPGLGMRLDRSDLNLLYRSRYPREIPDAYERLLLDAIEGERRLFIRSDELDAAWDLFTPALKELEEKKIIPELYPYGSRGPVGAHYLASKYNVRWGDLGEA

SEQ ID NO: 7 - DNA, 2082 bp, Arabidopsis thaliana
  source 1..2082: mol_type = other DNA
gtgagagataagaaatatcgaaaactatataataaaactactgttgtggataatgtaagagacgatacaaatagcctcataaggcaaaaaacaatggcatcatcatcatcatcacatttgttttgacgctccaagaaaatttgcatggcgacacattctatgatcattccttctccttcttcttcttcctcgtctctcgctactgctgcgtctccgtttaaagagactctaccactctttagtagatctctcacattcccgagaaaatccttgttttctcaggttcgcttgagattcttcgccgagaaacattctcagcttgatacttcaaatgggtgtgccactaatttcgcttctttacaagattctggagatcaacttacagaggaacatgttacaaaaggagagtctactcttagtattacagttgttggagcgtcaggggacttagccaagaagaagatatttcctgcattgtttgctctcttttatgagggttgtcttcctcaggacttttctgtttttggttatgctcggactaaactcactcatgaagagcttcgtgacatgattagtagtactttaacttgccggattgatcagagggaaaaatgtggggataagatggagcagttcttgaaaagatgcttttaccattcgggtcagtataactcggaagaggattttgcggaactaaacaagaagctcaaagagaaagaggcaggaaagatatcaaacaggctgtactatttgtcgatacccccaaacatattcgttgatgtggttaggtgtgcaagtcttagagcctcctcagagaatggctggacaagggtcattgtggaaaagccatttggtcgtgactctgaatcatccggagagttaactagatgtctgaaacagtatcttacagaggagcaaatcttcaggattgatcactatttgggaaaggagcttgttgagaacctttcagtgctccgattctcaaatcttgttttcgagcctctgtggtcaagaaattacatacgcaacgttcagctgattttttctgaggatttcggaacagaagggcgtggaggttactttgatcaatatggaatcatacgagacatcatgcaaaaccatctcttgcaaatacttgcactttttgcaatggagacgccagtgagcttagatgctgaggacataagaagtgaaaaggtgaaagttttgagatcaatgaaaccgttgcggcttgaagatgtggttgttggtcaatacaaaggccataacaagggaggaaaaacttatccaggttatacagacgatccaacggttccaaatcacagtctcactcctacatttgctgcagcagccatgttcatcaacaatgcgagatgggacggtgttccttttcttatgaaagctggaaaagctctccataccagaggagcagagatcagagttcagttcagacatgtcccaggaaacttgtacaagaaaagttttgctactaatttagacaatgcaacaaatgaactagtgattagagttcagcctgatgaagggatatacctgaggatcaacaacaaagttcctggtcttggaatgagactagatcgcagcgaccttaaccttctatatcgatccaggtatccaagagagataccagatgcatatgaaagactgcttctcgatgcaatcgaaggagagaggaggttgtttatcagaagtgatgagctagatgcagcatgggatctattcacaccggctttgaaggaactagaggagaagaaaatcattcctgagctctatccatatggaagcagaggtcctgttggtgcacattaccttgcatctaagtacaatgtgagatggggagatcttggagaagcttaagctttcgacagaaacaatatgatttggtgttaatatgagtaaactaacttgtcgatattatcaatgcgtagataaatccttgttaccaaaacttgctttttctctgacattttttgattgtttttggttctaaatcgagcatttcggaggaatgtaccaaaactcttgcaggaatagaccatttccatcattgcaacaggtttagag

SEQ ID NO: 8 - protein, 551 aa, Arabidopsis thaliana
MEGVGLRAVGSHCSLSEMDDLDLTRALDKPRLKIERKRSFDERSMSELSTGYSRHDGIHDSPRGRSVLDTPLSSARNSFEPHPMMAEAWEALRRSMVFFRGQPVGTLAAVDNTTDEVLNYDQVFVRDFVPSALAFLMNGEPDIVKHFLLKTLQLQGWEKRVDRFKLGEGVMPASFKVLHDPIRETDNIVADFGESAIGRVAPVDSGFWWIILLRAYTKSTGDLTLSETPECQKGMKLILSLCLAEGFDTFPTLLCADGCSMIDRRMGVYGYPIEIQALFFMALRSALSMLKPDGDGREVIERIVKRLHALSFHMRNYFWLDHQNLNDIYRFKTEEYSHTAVNKFNVMPDSIPEWVFDFMPLRGGYFVGNVGPAHMDFRWFALGNCVSILSSLATPDQSMAIMDLLEHRWAELVGEMPLKICYPCLEGHEWRIVTGCDPKNTRWSYHNGGSWPVLLWQLTAACIKTGRPQIARRAVDLIESRLHRDCWPEYYDGKLGRYVGKQARKYQTWSIAGYLVAKMLLEDPSHIGMISLEEDKLMKPVIKRSASWPQL

SEQ ID NO: 9 - DNA, 3061 bp, Arabidopsis thaliana
  source 1..3061: mol_type = genomic DNA
ccttcttcttccctctcctataaatctctccgtcttctctcttctctcatttcacgtcggagatcattttctcctcctccgcctccgcctccaccgccaccgccaccgccgccgccgacgacttttctcacttactcaacaccagaggtctctctctcactgtttcttcttctcctttctttcgatttcgtttggtagatttttagatctgatttttttctggtctctgtagctttacttgaagttgactctgtttctcgttatagatcttgatctgtttatgatttcgaataagaacagcttcaagcataattttatattcgaataaaaattagatgcaatgtgtttatttcgcgtgttccaactacacgagttttttttttttttttggattaggagttataacaatctcattgatcttattgttttgattttagatcttggtttagtgttataatggaaggtgttggactaagagctgtaggatcacactgttctttatctgagatggatgatcttgatttgactcgagctttggataaaccaagattgaagatcgaaaggaagagatcgtttgatgaaagatcgatgagtgagctatcgactggttacagtagacatgacggtatacacgattcgccgcgaggtagatcggtgcttgatacgcctctttcttctgctagaaactcctttgagcctcatcctatgatggctgaagcttgggaggctttaagaaggtcaatggtcttcttccgtggtcaacctgttggtactcttgccgcggttgataatactaccgatgaagtcttgaactatgatcaggtaaaactatcgaaaagttttcatggttttatctcaaaacgtttcaaaatgttttttttgcggttgttgtaggtgtttgtgagggattttgtaccgagtgcgttggcgtttctgatgaatggagaaccggatatagtgaagcattttttgcttaagacacttcagctgcaaggttgggagaaacgtgtggatcggtttaagttaggagaaggtgtgatgcctgcaagtttcaaggtgcttcatgatcctatccgtgaaacggataacattgttgcggatttcggtgagagtgctattggacgtgtggctcctgtagattcagggttctggtggattattcttcttcgtgcttataccaaatctactggagatttgactctctctgagacaccagagtgtcaaaagggaatgaaactgatcttgtctttgtgcttagctgaagggtttgacacgtttcctacactgctttgtgctgatggatgttccatgattgatcgaagaatggtattaaaagcaatctttgatcgaaactgattgttgtttcttgttttgtttggctattaagattgatttcttttttttcctgtatttgaagggtgtttatgggtatcccattgagatccaagcgttgttcttcatggctttgagatctgctttgtcgatgttaaagcccgatggagatggcagagaggtcattgagaggatcgttaagcgacttcatgccttgagcttccatatgcgcaattacttctggcttgatcatcagaacctcaatgacatttacaggtatagttatttcatttgctttagatgatatgtagagtcacaaaagatcactataacttatagtacatttgtttaaaaaggttcaagactgaggaatactctcacacggcggtgaacaagttcaatgtgatgcctgattctatacccgagtgggttttcgacttcatgcctctccgtggaggctactttgtgggaaacgtaggacctgcccatatggatttccggtggttcgcactcggtaattgtgtctccatactttcttcattggccactccagatcaatccatggccattatggacctccttgagcaccggtgggcagagcttgtaggtgagatgcctctcaagatttgttatccatgcctcgagggccatgagtggcgcatcgttactggctgtgaccccaagaacacacggtggagctaccacaatggtggatcttggccaggtcttttttctttccctggagactgattgcatgagacatttcttatctcgcttaactgagattttcgctagacctagccattagggtttttttcggtttgatttgggtttcaatttttttttgtagactggcataagaacagaaatattcatttaagatagtttagctagattttagttataaaaactcagtacttagtttagctacagtttggttgtggtttggattattcatatgaacgtatagatacattttagtttggttcaacttttgtatcttatgatcgcatttctttcttaaatatatacatttgtggcaacctttgatatattttgttttgaaatgttttgatagattgaataatgaacatcatgttgtgtagtgttaaatttgtcaataaaattgaatacagaatctgaccgcctttttgtttctctgctcaatgtacagttttgctgtggcagctaacagcagcgtgcattaagacagggagaccgcagatcgcaagacgcgcggttgacctcatagaatcgcgtcttcaccgagactgttggccagagtattacgacggtaagctcggaaggtacgttggaaaacaggcaaggaaataccagacttggtcaatcgcaggttacttagtggcgaaaatgttgctagaagatccttcacacattggtatgatctctcttgaagaagacaaactcatgaaaccagtcatcaagcgatctgcgtcttggccacaactctgaaaaaccgctttaggtttttgttccggttctaatatttgccacgatatgattttttgttgttgttgtttgtatgataatgttattgaaaacttgtgcggggacatagactttttattgattgctttctttcttgctttgcaagtaaggaaaaagaaaatgggaaaagctcgtttgccgtttttatcgaaattacacctttttttttaataatttgcattttggcgctattgcaatatgaatatctttcaataagctg

(19) United States
(12) Patent Application Publication
(10) Pub. No.: US 2026/0308608 A1
(43) Pub. Date: Oct. 8, 2026

(54) Modified Plant and Methods of Producing and Using the Same to Improve Agronomic Traits

(71) Applicant: The Board of Trustees of the University of Illinois, Urbana, IL (US)

(72) Inventor: Ken N. Paige, Monticello, IL (US)

(21) Appl. No.: 19/634,468

(22) Filed: Mar. 31, 2026

Related U.S. Application Data
(60) Provisional application No. 63/783,353, filed on Apr. 4, 2025.

(51) Int. Cl. C12N 15/82; C07K 14/415

(52) U.S. Cl. CPC C12N 15/8261; C07K 14/415

(57) ABSTRACT

Modified plants and a method of using the same to improve one or more agronomic traits are provided, wherein the modified plant or a plant part thereof comprises an endogenous or exogenous nucleic acid encoding an Increased Level of Polyploidy 1 (ILP1) polypeptide. Methods of producing a modified plant, and products made from the modified plant, are also provided.

### REFERENCE TO RELATED APPLICATIONS

This application claims the benefit of priority from U.S. Provisional Application Ser. No. 63/783,353, filed Apr. 4, 2025, the content of which is incorporated herein by reference in its entirety.

### REFERENCE TO AN ELECTRONIC SEQUENCE LISTING

The contents of the electronic sequence listing (name: IL0048US_ST26.xml; size: 28,092 bytes; and date of creation: Mar. 28, 2026) is herein incorporated by reference in its entirety.

### BACKGROUND OF THE INVENTION

With the global population continuing to increase, and suitable agricultural areas expected to decrease, increases in food production will become increasingly important. Several approaches, including technologies to increase food production through intensification rather than expansion, reducing food waste, and changes in diet, among others, have been proposed. However, there have been few significant increases in yield demonstrated in major food crops such as wheat, rice, potato, corn, soybeans, sorghum, cassava barley, sweet potatoes, or pulses (lentils, beans, peas). New strategies to increase food production are needed to meet the growing global demand and to address the challenges of climate change, land and water use constraints, and biodiversity loss.

Plants have evolved a variety of mechanisms to mitigate the negative effects of herbivory. One of these mechanisms is the ability to compensate for tissue loss by regrowth. In some instances, plants can respond to plant herbivory with an increase in plant reproductive success (a specialized case of tolerance, termed “overcompensation,” i.e., increased flower, fruit and seed production). Overcompensation was first demonstrated in Ipomopsis aggregata, where a 2.4-fold increase in seed yield was observed following the removal of 95% of the aboveground biomass of this monocarpic biennial by mule deer and elk.

It has also been shown that following the removal of apical dominance, increases in ploidy level via endoreduplication lead to rapid regrowth and an increase in reproductive success, further explaining, in part, the phenomenon of overcompensation in plants. Endoreduplication is the replication of the genome without mitosis, which leads to endopolyploidy, an increase in cellular chromosome number. Screening of dominant mutants of Arabidopsis thaliana identified the Increased Level of Polyploidy 1 (ILP1) gene as providing for increased polyploidy levels in first leaves (Yoshizumi et al. (2006) Plant Cell 18(10):2452-2468). By overexpressing ILP1, a positive regulator of endoreduplication, and thus compensation, glucosinolate production and tolerance in the Col-0 genotype of A. thaliana has been demonstrated (Mesa et al. (2017) Ecology 98 (10):2528-2537).

### SUMMARY OF THE INVENTION

Provided herein is a modified plant or plant part thereof comprising a nucleic acid encoding an Increased Level of Polyploidy 1 (ILP1) protein, wherein the nucleic acid comprises: (a) a protein-coding sequence encoding the ILP1 protein, and (b) one or more regulatory elements operably linked to the protein-coding sequence.

Also provided is a method for improving one or more agronomic traits of a plant or plant part thereof comprising a nucleic acid encoding an ILP1 protein, wherein the nucleic acid comprises: (a) a protein-coding sequence encoding the ILP1 protein, and (b) one or more regulatory elements operably linked to the protein-coding sequence, comprising killing or inhibiting the growth of a sufficient number of cells in the shoot apical meristem of the modified plant thereby improving one or more agronomic traits of the plant.

### BRIEF DESCRIPTION OF THE DRAWINGS

FIG. 1 provides an example expression construct encoding ILP1.

FIGS. 2A-2C provide seed number (FIG. 2A), seed weight (FIG. 2B), and seed yield (FIG. 2C) for five different modified lines of soybeans including modified lines 1 (indicated as 1 and 1-32), 10 (indicated as 10 and 10-9), 31, 33, and 40 as compared to wild-type Williams 82 (Will82). Asterisks show significance p<0.05. Error bars are standard error.

FIGS. 3A-3B provides percent changes of phenotypes associated with endoreduplication for modified lines 33 and 40 (FIG. 3A) and Cell Cycle Value for modified line 33 (FIG. 3B) as compared to wild-type Williams 82 (Will82). Asterisks denote significance (p<0.05) and plus signs denote marginal significance (p<0.1). Note that in FIG. 3B, the herbicide-sensitive plants showed the same relationship for endoreduplication as the wild-type, whereas the resistant unclipped plants have a significantly higher level of endoreduplication over the unclipped control.

FIGS. 4A-4B provide protein (FIG. 3A) and oil (FIG. 3B) concentrations for modified lines of soybeans including modified lines 1 (indicated as 1 and 1-32), 10 (indicated as 10 and 10-9), 31, 33, and 40 as compared to wild-type Williams 82 (Will82). Error bars represent the standard error of the mean.

### DETAILED DESCRIPTION OF THE INVENTION

The following descriptions and examples illustrate embodiments of the present disclosure in detail. Although the present disclosure has been described in some details by way of illustration and example for purposes of clarity and understanding, it will be apparent that certain changes and modifications can be practiced within the scope of the appended claims.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular aspects described herein and as such can vary. Those of skill in the art will recognize that there can be variations and modifications of the present disclosure, which can be encompassed within its scope.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All terms are intended to be understood as they would be understood by a person skilled in the art. Unless defined otherwise, all technical and scientific terms used herein have the same meaning as commonly understood by one of ordinary skill in the art to which the disclosure pertains.

In this application, the use of the singular includes the plural unless specifically stated otherwise. It must be noted that, as used in the specification, the singular forms “a,” “an,” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean A individually; B individually; C individually; A and B; B and C; A and C; and A, B, and C. The term “or” can be used conjunctively or disjunctively unless the context specifically refers to a disjunctive use.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any aspect described herein can be implemented with respect to any method or composition herein, and vice versa. Furthermore, compositions of the present disclosure can be used to achieve the methods described herein.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed. The term “under suitable condition” or “under suitable reaction condition” refers to any environment that permits a desired reaction to take place.

The present invention is based, in part, on the discovery that overexpression of ILP1 in an oilseed plant results in an increase in the level of endopolyploidy leading to an increase in one or more agronomic traits, e.g., seed yield. Further, inhibiting the growth of or killing a significant number of cells in the shoot apical meristem, without significantly damaging growth of other parts (e.g., stems, leaves, or roots) of the plant, may be used to further increase yield traits in plants overexpressing ILP1.

Accordingly, provided herein is a modified plant or plant part thereof comprising a nucleic acid encoding an ILP1 protein, wherein the nucleic acid comprises: (a) a protein-coding sequence encoding the ILP1 protein, and (b) one or more regulatory elements operably linked to the protein-coding sequence, as well as methods of producing the modified plant and using the modified plant to improve one or more agronomic traits of a plant. As used herein, a “modified” plant or plant part thereof refers to a plant or plant part thereof wherein all or a portion of the nucleic acid of the disclosure has been artificially incorporated into a host cell's genome or all or a portion of an endogenous nucleic acid has been mutated or changed by means of targeted alteration such as by CRISPR-Cas targeted modification.

In some aspects of the nucleic acid of the disclosure, the protein-coding sequence encoding the ILP1 protein and/or the one or more regulatory elements are exogenous to the modified plant or plant part thereof. The term “exogenous” refers to any material that is introduced into or produced outside of a cell or organism. In accordance with this aspect, an exogenous nucleic acid comprising a heterologous promoter, e.g., a constitutive or inducible promoter, operably linked to an ILP1 coding sequence is introduced into a plant or plant part thereof to modulate expression of ILP1 in the plant or plant part thereof.

As used herein, a “plant part” refers to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, collenchyma cells, sclerenchyma cells, stomates, guard cells, cuticle, mesophyll cells; callus tissue; and cuttings of a plant. In some aspects, a plant part is a fruit or seed. The term “plant part” also includes plant cells, including plant cells that are intact in plants and/or parts of plants, plant protoplasts, plant tissues, plant organs, plant cell tissue cultures, plant calli, plant clumps, and the like.

Accordingly, it is to be understood that as used herein the term “modified” includes any cell, cell line, callus, tissue, plant part or plant the genotype of which has been altered by the presence of a transgene, or a targeted alteration in nucleic acid (e.g., using CRISPR-Cas or other means), as well as those created by sexual crosses or asexual propagation from the initial modified plant.

In some aspects, all or a portion of a nucleic acid (transgene) may be heterologous to a plant or plant part thereof. In some aspects, a nucleic acid may comprise, consist of, or consist essentially of a heterologous protein-coding sequence encoding a protein of interest. In some aspects, a nucleic acid may comprise, consist of, or consist essentially of a heterologous protein-coding sequence encoding a protein of interest, e.g., ILP1, wherein the protein-coding sequence is operably linked to one or more heterologous regulatory elements such as promoter, e.g., a heterologous promoter. In some aspects, a nucleic acid may comprise, consist of, or consist essentially of a heterologous promoter.

As used herein, “heterologous” refers to a nucleic acid/protein that originates from a foreign species (i.e., a different species as the modified plant), or, if from the same species, is substantially modified from its native form in composition and/or genomic locus by deliberate human intervention. In some aspects, a heterologous sequence, e.g., heterologous promoter, is a sequence obtained from a species of plant that is different from the modified plant.

As used herein, a “promoter” is a regulatory element that controls, modulates, or regulates the transcription of a nucleic acid of interest (e.g., a protein-coding sequence) that is operably linked the promoter. Typically, a “promoter” refers to a nucleic acid that contains a binding site for RNA polymerase II and directs the initiation of transcription. In general, promoters are found 5′, or upstream, relative to the start of a protein-coding sequence. A promoter may comprise other elements that act as regulators of gene expression, e.g., a promoter region. These include a TATA box consensus sequence, and often a CAAT box consensus sequence (Breathnach & Chambon (1981) Annu. Rev. Biochem. 50:349). The CAAT box may be substituted by the AGGA box (Messing et al. (1983) in Genetic Engineering of Plants, Kosuge et al. (eds.) Plenum Press, pp. 211-227).

Promoters useful herein may include, for example, constitutive, inducible, temporally regulated, developmentally regulated, chemically regulated, tissue-preferred and/or tissue-specific promoters. In some aspects, a heterologous promoter is a promoter that is functional in a plant cell. In some aspects, a heterologous promoter is a constitutive promoter. In some aspects, a heterologous promoter is an inducible promoter.

Examples of constitutive promoters useful for expression in plants include, but are not limited to, a cauliflower mosaic virus (CaMV) promoter such as the CaMV 35S promoter (Odell et al. (1985) Nature 313:810-812) or CaMV 19S promoter (Lawton et al. (1987) Plant Mol. Biol. 9:315-324), nos promoter (Ebert et al. (1987) Proc. Natl. Acad. Sci USA 84:5745-5749), Adh1 promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), α-tubulin promoter, actin promoter such as the rice actin 1 promoter (Wang et al. (1992) Mol. Cell. Biol. 12:3399-3406, U.S. Pat. No. 5,641,876), a GmScream promoter such as those derived from GmScreamM1, GmScreamM4, GmScreamM8 genes (Zhang et al. (2015) Plant Science 241:189-198) or GmubiXL genes (De La Torre & Finer (2015) Plant Cell Reports 34:111-120), or ubiquitin promoter such as the Arabidopsis ubiquitin 10 promoter. In some aspects, the CaMV 35S promoter is used to modulate expression of a nucleic acid encoding ILP1.

The term “inducible promoter” refers to a promoter that selectively expresses a coding sequence in response to the presence of an endogenous or exogenous stimulus, such as by a chemical compound (chemical inducer), or in response to environmental, hormonal, chemical, and/or developmental signals. Inducible or regulatable promoters include promoters which are induced or regulated, for example, by light, heat, stress, flooding or drought, salt stress, osmotic stress, plant hormones, wounds or chemicals such as ethanol, abscisic acid (ABA), jasmonate, salicylic acid or safeners. Examples of inducible promoters useful for expression in plants include, but are not limited to, the promoter of the nitrate reductase gene (Pnr), and the promoter of duplicated carbonic anhydrase gene 1 (Pdca1). In some aspects, expression of ILP1 by a modified plant may be induced before, after, or at same time as killing or inhibiting the growth of the shoot apical meristem of the modified plant.

Examples of tissue-preferred and/or tissue-specific promoters useful for expression in plants include, but are not limited to, cab promoter (Sullivan et al. (1989) Mol. Gen. Genet. 215:431-440), PEPCase promoter (Hudspeth & Grula (1989) Plant Mol. Biol. 12:579-589), R gene complex-associated promoter (Chandler et al. (1989) Plant Cell 1:1175-1183), poplar xylem-specific secondary cell wall specific cellulose synthase promoter, or phaseolin promoter.

Additional regulatory elements useful herein include, but are not limited to, introns, enhancers, 5′- and 3′-untranslated regions and/or termination sequences, e.g., termination sequences derived from ILP1, another plant gene, or from T-DNA. The terminator to be added may be derived from, for example, the nopaline synthase or octopine synthase genes, or alternatively from another plant gene.

In some aspects, expression of an endogenous or exogenous ILP1 protein-coding sequence is modulated by operably linking a heterologous promoter to the coding sequence of ILP1. As used herein, “modulate, “modulating,” or grammatical variations thereof, refers to an increase or decrease in the levels of ILP1 protein and/or mRNA encoding the same, and/or regulating the expression of ILP1 protein and/or ILP1 mRNA is regulated in a manner that is different from that of the endogenous ILP1 gene. In some aspects, a heterologous promoter provides for overexpression of the ILP1 protein and/or ILP1 mRNA in a plant or plant part thereof, i.e., a 1.2-fold, 1.5-fold, 2-fold, 3-fold, 4-fold, 5-fold, or 10-fold increase, as compared to a plant or plant part thereof that is devoid of the heterologous promoter operably linked to the ILP1 nucleic acid. In some aspects, a heterologous promoter provides for constitutive expression of the ILP1 protein and/or ILP1 mRNA in a plant or plant part thereof. In some aspects, a heterologous promoter provides for inducible expression of the ILP1 protein and/or ILP1 mRNA in a plant or plant part thereof.

“Operably linked” or “operably associated,” as used herein in reference to nucleic acids, it is meant that the indicated elements are functionally related to each other and are also physically related. Thus, the term “operably linked” or “operably associated” as used herein, refers to nucleotide sequences on a single nucleic acid molecule that are functionally associated. Thus, a first nucleic acid sequence that is operably linked to a second nucleic acid sequence means a situation when the first nucleic acid sequence is placed in a functional relationship with the second nucleic acid sequence. For instance, a promoter is operably associated with a nucleic acid sequence if the promoter effects the transcription or expression of said nucleic acid sequence. Those skilled in the art will appreciate that the control sequences (e.g., promoter) need not be contiguous with the nucleic acid sequence to which it is operably associated, as long as the control sequences function to direct the expression thereof. Thus, for example, intervening untranslated, yet transcribed, nucleic acid sequences may be present between a promoter and the nucleic acid sequence, and the promoter may still be considered “operably linked” to the nucleic acid sequence.

In some aspects, a heterologous promoter is operably linked to and modulates expression of an endogenous nucleic acid encoding ILP1. The term “endogenous” refers to a molecule, e.g., nucleic acid or polypeptide, which is naturally present in a cell or organism. In accordance with this aspect, a heterologous promoter, e.g., a constitutive or inducible promoter, is introduced upstream of the endogenous ILP1 coding sequence to replace the endogenous ILP1 promoter.

In some aspects, all or a portion of a nucleic acid (transgene) may be native or endogenous to a plant or plant part thereof. In accordance with some aspects, a plant or plant part thereof is modified when one or more copies of a native or endogenous nucleic acid encoding an ILP1 protein are introduced into the plant or part thereof thereby increasing the gene copy number of the native or endogenous nucleic acid encoding the ILP1 protein. In some aspects, a nucleic acid may comprise, consist of, or consist essentially of a native or endogenous protein-coding sequence encoding a protein of interest. In some aspects, a nucleic acid may comprise, consist of, or consist essentially of a native or endogenous protein-coding sequence encoding a protein of interest, e.g., ILP1, wherein the protein-coding sequence is operably linked to one or more native or endogenous regulatory elements such as promoter, e.g., a native or endogenous promoter. In some aspects, a nucleic acid may comprise, consist of, or consist essentially of a native or endogenous promoter.

As used herein, a “native” or “endogenous” nucleic acid refers to a nucleic acid that occurs naturally in a plant genome, and retains its endogenous gene structure, including the full protein-coding sequence (open reading frame), and its associated regulatory elements, such as promoter, untranslated regions (UTRs), introns (if present), and terminator sequences, and has not been modified by codon optimization, sequence engineering, or synthetic redesign. In some aspects, a nucleic acid of the disclosure is a native or endogenous ILP1 gene. Excluded from the description of a native or endogenous nucleic acid is an Arabidopsis plant genetically engineered with a native nucleotide sequence encoding an ILP1 protein.

In some aspects, all or a portion of a nucleic acid of the disclosure is from a dicotyledonous (dicot) plant. In some aspects, all or a portion of a nucleic acid of the disclosure is from a monocotyledonous (monocot) plant.

In some aspects, a method of producing a modified plant is provided, the method comprising the steps of introducing into at least one plant cell a nucleic acid encoding an ILP1 protein operably linked to one or more regulatory elements (e.g., a heterologous promoter); and regenerating a plant from the at least one plant cell thereby producing a modified plant. As used herein, the term “introducing” or “transforming” means presenting a nucleic acid of interest to the plant, plant part, or plant tissue in such a manner that the nucleic acid gains access to the interior of a cell. Where more than one nucleic acid of interest is to be introduced, these nucleic acids may be assembled as part of a single nucleic acid or DNA construct, or as separate nucleic acids or DNA constructs, and may be located on the same or different transformation vectors. Accordingly, these nucleic acids may be introduced into plant cells in a single transformation event, in separate transformation events, or, e.g., as part of a breeding protocol.

A modified plant may be produced by transformation of a plant cell with a nucleic acid of interest, including an expression construct that includes a nucleic acid of interest, the regeneration of a population of plants resulting from the insertion of the transferred nucleic acids into the genome of the plant and selection of a plant characterized by insertion into a particular genome location. Insertion of the transferred nucleic acids may be characterized phenotypically by the expression of the inserted nucleic acids. At the genetic level, insertion of the transferred nucleic acids may be identified by observing that the transferred nucleic acids become a part of the genetic makeup of a plant. Thus, insertion of a transferred nucleic acids may be confirmed via PCR, Southern hybridization, northern hybridization, western blot analysis, or other means. For example, DNA may be prepared from a modified plant, an ILP1 gene-specific primer may be designed, and PCR may be carried out. The PCR amplicon may then be subjected to agarose gel electrophoresis, polyacrylamide gel electrophoresis, or capillary electrophoresis and stained with, e.g., ethidium bromide thereby allowing detection of the amplicon as a band. Further, protein may be extracted from the plant cell, two-dimensional electrophoresis may be carried out to fractionate the protein, and a band corresponding to the ILP1 protein may be detected. A modified plant also refers to progeny produced by a sexual cross between the transformant and another plant wherein the progeny include the nucleic acid of interest.

Transformation protocols as well as protocols for introducing a nucleic acid of interest, including an expression construct that includes a nucleic acid of interest, into plants may vary depending on the type of plant or plant cell, i.e., monocot or dicot, targeted for transformation. Suitable methods of introducing nucleic acids into plant cells and subsequent insertion into the plant genome include electroporation (U.S. Pat. Nos. 5,384,253 and 5,472,869; Dekeyser et al. (1990) Plant Cell 2:591-602); direct DNA transfer to plant cells by PEG precipitation (Hayashimoto et al. (1990) Plant Physiol. 93:857-863); direct DNA transfer to plant cells by microprojectile bombardment (McCabe et al. (1988) Bio/Technology 6:923-926; U.S. Pat. Nos. 5,489,520; 5,538,877; and 5,538,880) and DNA transfer to plant cells via infection with Agrobacterium. Methods and compositions for rapid plant transformation are also found in US 2017/0121722, herein incorporated by reference in its entirety. Methods such as microprojectile bombardment or electroporation may be carried out with “naked” DNA where the nucleic acid of interest may be simply carried on any E. coli-derived plasmid cloning vector. In the case of viral vectors, it is desirable that the system retain replication functions but lack functions for disease induction. A nucleic acid of interest may also be introduced into the genome of a plant cell using double-stranded break technologies such as TALENs, meganucleases, zinc finger nucleases, CRISPR-Cas, and the like.

In some aspects, the method herein further includes the step of regenerating a plant from a plant cell. As used herein, “regenerate,” “regeneration,” and “regenerating” (and grammatical variations thereof) means formation of a plant from various plant parts (e.g., plant explants, callus tissue, plant cells) that includes a rooted shoot. The regeneration of plants from various plant parts is well known in the art. See, e.g., Methods for Plant Molecular Biology (Weissbach et al., eds. Academic Press, Inc. (1988). For example, regenerating plants containing a construct introduced by Agrobacterium from leaf explants can be achieved as described by Horsch et al. (1985) Science 227:1229-1231. Briefly, transformants are grown in the presence of a selection agent and in a medium that induces the regeneration of shoots. See, for example, Fraley et al. (1983) Proc. Natl. Acad. Sci. USA 80:4803-4807. This method typically produces shoots within about two weeks to four weeks, and the transformed shoots are then transferred to an appropriate root-inducing medium containing the selective agent and an antibiotic to prevent further bacterial growth. Typically, transformed shoots that root in the presence of the selective agent to form plantlets are then transplanted to soil or other media to allow the production of additional roots.

Modified plantlets may then be propagated in soil or a soil substitute to promote growth into a mature modified plant. Propagation of modified plants from these plantlets is performed, e.g., in perlite, peatmoss and sand (1:1:1) or commercial plant potting mix under glasshouse conditions. These plants may then be grown, and either pollinated with the same transformed strain or different strains, and the resulting progeny having expression of the desired phenotypic characteristic. Two or more generations may be grown to ensure that expression of the desired phenotypic characteristic is stably maintained and inherited and then seeds harvested to ensure expression of the desired phenotypic characteristic has been achieved, in this manner, the present disclosure provides transformed seed (also referred to as “modified seed”) having a nucleic acid of the disclosure stably incorporated into its genome.

In one aspect of the plants and methods herein, a heterologous promoter is introduced into the genome of a plant to replace the endogenous ILP1 promoter. In accordance with this aspect, the nucleic acid encoding the ILP1 protein is an endogenous nucleic acid. Introduction of a heterologous promoter into a plant genome to replace an endogenous promoter may be carried out using any conventional genome editing method and composition to target and cleave genomic DNA in a site-specific manner (e.g., using nucleases such as Zinc Finger Nucleases (ZFNs), Meganucleases, Transcription Activator-Like Effector Nucleases (TALENS) and Clustered Regularly Interspaced Short Palindromic Repeats (CRISPR)-associated nucleases with an engineered crRNA/tracr RNA). By way of illustration, the CRISPR/Cas9 genome editing system has been used in a wide variety of organisms, including monocotyledonous and dicotyledonous plants. One or more single guide RNAs (sgRNAs) with target sequences homologous to the desired gene to be edited are introduced with Cas9 nucleases and used to direct Cas9 proteins to specific genomic sites (Liu (2016) Curr. Protocol. Mol. Biol. 30:70-7). Other double-stranded nucleases such as Cms1 may also be used (U.S. Pat. No. 9,896,696). Similarly, TALENs have been used to modify economically important food crops and biofuels. TALENs may be used to introduce a targeted fragment of DNA that is homologous to the cleavage site in order to specifically introduce a heterologous sequence into a locus using homologous recombination.

In another aspect of the plants and methods herein, the nucleic acid encoding the ILP1 protein is a heterologous nucleic acid operably linked to a heterologous promoter. Introduction of a heterologous promoter operably linked to a heterologous ILP1 nucleic acid into a plant or part thereof may be carried out using any conventional plant transformation method known in the art and/or described herein. For instance, a genetic construct may be prepared to facilitate introduction and/or expression of the nucleic acid sequences described herein in a plant. Thus, in some aspects, the invention provides a construct comprising a heterologous promoter operably linked to an ILP1 nucleic acid. The term “construct,” “recombinant DNA construct,” “recombinant construct,” “expression construct,” or “expression cassette” refers to any agent such as a plasmid, cosmid, virus, BAC (bacterial artificial chromosome), autonomously replicating sequence, phage, or linear or circular single-stranded or double-stranded DNA or RNA nucleotide sequence, derived from any source, capable of genomic integration or autonomous replication. According to some aspects, a modified plant comprising a heterologous promoter operably linked to an ILP1 nucleic acid is referred to as a “transgenic plant.”

The nucleotide sequence introduced into the plant must comprise a sequence encoding an ILP1 protein (Increased Level of Ploidy 1) or functional equivalent, and the regulatory elements naturally associated with that ILP1 gene in the source plant. Such sequences may be derived from the same species (cisgenic or intragenic transformation) or derived from a different plant species (transgenic transformation), including both dicots and monocots. In monocot plants, nucleic acids encoding ILP1 may be referred to in the scientific literature as ILP1 homologs or ILP1 orthologs and may be annotated as “C2ORF3-like” or “spliceosome-associated” proteins. A homolog is a gene that shares a common evolutionary ancestor with another gene. Homologous genes may occur within the same species or across different species and may or may not retain the same function. An ortholog is a specific type of homolog that occurs in different species and arose from a common ancestral gene through speciation. Orthologs typically retain similar biological functions across species. Accordingly, monocot nucleotide sequences that encode proteins evolutionarily related to Arabidopsis ILP1 may be referred to as ILP1 homologs or orthologs, depending on their evolutionary relationship and are intended to be included within the use of the term “ILP1” unless otherwise specified. These sequences, when native and including their endogenous regulatory elements, may be used to transform plants to modulate ILP1-associated biological processes such as RNA splicing, cell cycle regulation, or endoreduplication.

In aspects of the modified plant or plant part and methods for producing the same, the ILP1 protein may comprise an amino acid sequence having at least about 70% sequence identity to SEQ ID NO:1 and/or SEQ ID NO:2, e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1 and/or SEQ ID NO:2.

Examples of ILP1 proteins comprising an amino acid sequence having at least about 70% sequence identity to SEQ ID NO:1 and/or SEQ ID NO:2 are provided in Table 1.

TABLE 1

| Plant | GENBANK Accession No. | % Identity |
|---|---|---|
| Camelina sativa | XP_010423056.1 | 88%^{a} |
| Capsella rubella | XP_023635988.1 | 89%^{a} |
| Brassica rapa | XP_009122469.1 | 80%^{a} |
| Brassica napus | XP_048625102.1 | 79%^{a} |
| Cardamine amara | KAL1196337.1 | 86%^{a} |
| Glycine soja | KAL5163232.1 | 99%^{b} |
| Glycine max | XP_006605552.1 | 92%^{b} |
| Phaseolus coccineus | KAK7376993.1 | 87%^{b} |
| Arachis hypogaea | KAL4307896.1 | 74%^{b} |

^{a}Sequence identity with SEQ ID NO: 1.
^{b}Sequence identity with SEQ ID NO: 2.

In some aspects, an ILP1 nucleic acid encoding an ILP1 protein may comprise a nucleotide sequence having at least 70% sequence identity to SEQ ID NO:3, SEQ ID NO:4, and/or SEQ ID NO: 5, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:3, SEQ ID NO:4, and/or SEQ ID NO:5.

With respect to nucleic acid and/or amino acid sequences herein, the term “identity” refers to the extent that sequences are identical on a nucleotide-by-nucleotide or amino acid-by-amino acid basis over a segment or window of comparison. A “percentage of sequence identity” is calculated by comparing two optimally aligned sequences over the window of comparison, determining the number of positions at which the identical nucleotide or amino acid residue is present in both sequences to yield the number of matched positions, dividing the number of matched positions by the total number of positions in the window of comparison, and multiplying the result by 100 to yield the percentage of sequence identity. Sequence identity may also be to a fragment or portion of the full-length nucleic acid or polypeptide. For example, a sequence may have 40% overall sequence identity with a full-length reference sequence, but a sequence of a particular region may have 90% or more sequence identity with the reference sequence.

Nucleic acid and/or polypeptide sequences having at least 95% sequence identity to the sequences herein may be, e.g., nucleic acids that have been codon optimized for expression in a plant of interest; a natural variant of a nucleic acid and/or polypeptide sequence such as a paralog or single nucleotide polymorphism; a mutation in a polypeptide sequence that increases activity; and/or a conservative amino acid substitution in a polypeptide sequence that does not substantially alter activity. Conservative amino acid changes are well-known in the art and may be selected in accordance with the properties of the 20 main amino acids.

In some aspects, any one of the modified plants or plant parts thereof and/or methods herein may further include introduction of a second heterologous promoter, wherein the second heterologous promoter is operably linked to and modulates expression of a nucleic acid encoding a glucose-6-phophospate dehydrogenase (G6DP) polypeptide. Accordingly, also provided herein is a modified plant or plant part thereof, including a modified oilseed plant, comprising (a) a first heterologous promoter, wherein the heterologous promoter is operably linked to and modulates expression of a nucleic acid encoding an Increased Level of Ploidy 1 (ILP1) polypeptide and (b) a second heterologous promoter, wherein the second heterologous promoter is operably linked to a glucose-6-phophospate dehydrogenase (G6DP) gene or an invertase gene.

Also provided herein is method of producing a modified plant, including a modified oilseed plant, comprising introducing into at least one plant cell (a) a heterologous promoter, wherein the heterologous promoter is operably linked to and modulates expression of a nucleic acid encoding an ILP1 protein, and (b) a second heterologous promoter, wherein the second heterologous promoter is operably linked to a glucose-6-phophospate dehydrogenase (G6DP) gene or a invertase gene; and regenerating a plant from the at least one plant cell thereby producing a modified plant.

Glucose-6-Phosphate Dehydrogenase 1 (G6PD1) catalyzes the rate-limiting first step of the oxidative pentose-phosphate pathway (OPPP), which plays a central role in generalized metabolism and biosynthesis. In addition to metabolic intermediates and the reductant NADPH, OPPP also supports the production of nucleotides and preferably endoreduplication, which contributes to compensatory performance through its likely effects on cell growth, gene expression, and metabolism. Accordingly, an increase in the expression of G6PD1 may be used to further increase overcompensation in a modified plant or plant part thereof as described herein.

In aspects of the modified plant or plant part and methods for producing the same, the G6PD1 polypeptide may comprise an amino acid sequence having at least about 70% sequence identity to SEQ ID NO:6, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6.

Examples of G6PD1 polypeptides comprising an amino acid sequence having at least about 70% sequence identity to SEQ ID NO:6 are provided in Table 2.

TABLE 2

| Plant | GENBANK Accession No. | % Identity |
|---|---|---|
| Capsella rubella | XP_006283407.1 | 94% |
| Cardamine amara | KAL1210216.1 | 94% |
| Camelina sativa | XP_019089820.1 | 94% |
| Brassica napus | XP_013715108 | 91% |
| Brassica rapa | XP_009108011 | 90% |

In some aspects, an G6PD1 nucleic acid encoding an G6PD1 polypeptide may comprise a nucleotide sequence having at least 70% sequence identity to SEQ ID NO:7, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:7.

In some aspects, any one of the modified plants or plant parts thereof and/or methods herein may further include introduction of a second heterologous promoter, wherein the second heterologous promoter is operably linked to and modulates expression of a nucleic acid encoding an invertase polypeptide. Invertases comprise a family of metabolic isoenzymes that catalyze the hydrolysis of sucrose into glucose and fructose. There are three forms of invertase: neutral/cytosolic, cell wall and vacuolar, that share similar catalytic function. In some aspects, the invertase polypeptide is Cytosolic Invertase 1. The glucose produced through sucrose hydrolysis is in part shunted to G6PD1 in the OPPP to produce ribulose-5-phosphate and erythrose-4-phosphate. These compounds in turn serve as intermediates for nucleotide synthesis and plant defensive chemistry through the shikimate pathway. Due to their catalysis of a reaction that directly feeds the rate-limiting step of the OPPP, invertases may therefore play an important role in plant compensation via damage through their integration with the OPPP and subsequent downstream metabolic processes. Accordingly, an increase in the expression of a cytosolic invertase may be used to further increase overcompensation in a modified plant or plant part thereof as described herein.

In aspects of the modified plant or plant part and methods for producing the same, the invertase polypeptide may comprise an amino acid sequence having at least 70% sequence identity to SEQ ID NO:8, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:8.

Examples of invertase polypeptides comprising an amino acid sequence having at least about 70% sequence identity to SEQ ID NO:8 are provided in Table 3.

TABLE 3

| Plant | GENBANK Accession No. | % Identity |
|---|---|---|
| Camelina sativa | XP_010461321.1 | 99% |
| Capsella rubella | XP_006304719.1 | 99% |
| Cardamine amara | KAL1213513.1 | 98% |
| Brassica rapa | XP_009107914.2 | 95% |
| Brassica napus | XP_048594463.1 | 95% |

In some aspects, an invertase nucleic acid encoding an invertase polypeptide may comprise a nucleotide sequence having at least 70% sequence identity to SEQ ID NO:9, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:9.

In some aspects, the second heterologous promoter is operably linked to a nucleic acid encoding a G6PD1 polypeptide. In some aspects, the second heterologous promoter is operably linked to an endogenous nucleic acid encoding a G6PD1 polypeptide. In some aspects, the second heterologous promoter is operably linked to a nucleic acid encoding an invertase polypeptide, e.g., a cytosolic invertase. In some aspects, the second heterologous promoter is operably linked to an endogenous nucleic acid encoding an invertase polypeptide, e.g., a cytosolic invertase. In some aspects, a modified plant or plant part thereof includes a heterologous promoter operably linked to a nucleic acid encoding a G6PD1 polypeptide and a heterologous promoter operably linked to a nucleic acid encoding an invertase polypeptide. In accordance with such an aspect, the modified plant or plant part thereof may include a second heterologous promoter operably linked to a nucleic acid encoding a G6PD1 polypeptide (e.g., an endogenous or exogenous G6PD1) and a third heterologous promoter operably linked to a nucleic acid encoding an invertase polypeptide (e.g., an endogenous or exogenous invertase). In some aspects, expression of ILP1 and expression of G6PD1 and/or invertase genes may be increased at the same time. As a result, the amount of G6PD1 and/or invertase enzyme and the amount of ILP1 protein are each increased in plant cells at the same time. Introduction of a second (and third) heterologous promoter may be carried out as described elsewhere herein. Similarly, introduction of a second (and third) heterologous promoter operably linked to an endogenous nucleic acid encoding the G6PD1 polypeptide or invertase polypeptide may be carried out as described elsewhere herein.

In some aspects, the heterologous promoter operably linked to a nucleic acid encoding ILP1 (i.e., the first heterologous promoter) may be the same or different from the second (and/or third) heterologous promoter operably linked to an endogenous nucleic acid encoding the G6DP polypeptide or invertase polypeptide. In some aspects, the first heterologous promoter and second (and/or third) heterologous promoter are constitutive promoters. In some aspects, the first heterologous promoter and second (and/or third) heterologous promoter are inducible promoters. In some aspects, the first heterologous promoter is inducible by a first stimulus and the second (and/or third) heterologous promoter is inducible by a second (and/or third) stimulus that is different than the first stimulus.

In some aspects, expression of the ILP1 protein in the modified plant or part thereof, and optionally G6DP polypeptide and/or invertase polypeptide, imparts an improvement in one or more agronomic traits to the modified plant or part thereof as compared to a plant or part thereof devoid of the heterologous promoter. As used herein an improvement in one or more agronomic traits means an improvement of at least one characteristic of a plant resulting from the introduction of one or more the nucleic acids described herein into the plant. Such traits include, but are not limited to, enhanced plant morphology, physiology, growth and development, yield, nutritional enhancement, disease or pest resistance, or environmental or chemical tolerance. In some aspects, the one or more agronomic traits comprise increased fitness, seed yield, drought tolerance, pest/pathogen resistance, disease resistance, seed pod yield, biomass yield, seed protein yield, seed oil yield, and/or seed starch yield.

As used herein, the term “seed yield” refers to a measurable number of seed (grain) produced by a population of plants. “Seed yield” may be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seed weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Seed yield may be determined on a dry weight or on a fresh weight basis, or typically on a moisture adjusted basis, e.g., at 15.5 percent moisture.

“Increased seed yield” means that the seed yield of the respective population of plants is increased by a measurable amount over the seed yield of the same population of plants produced under the same conditions, but devoid of a nucleic acid described herein (e.g., a heterologous promoter operably linked to a nucleic acid encoding an ILP1 protein). Increased seed yield may be characterized by, e.g., increased seed number and/or increased seed weight. In some aspects, improved or increased seed yield refers to enhanced yield of seeds in terms of either dry weight or fresh weight, or both. In one aspect, an increase in seed yield refers to increased harvestable seed yield. Seed yield in accordance with the methods herein may be calculated on a per plant basis or in relation to a specific area, seed yield per acre/square meter/or the like. In some aspects, seed yield may be calculated in relation to a hectare of land, i.e., seeds produced per hectare.

“Seed oil” refers to the oil extracted or extractable from seeds of an oilseed plant. Increased “seed oil yield” means that the amount or quantity of seed oil obtained from the respective population of plants is increased by a measurable amount over the amount or quantity of seed oil obtained from the same population of plants produced under the same conditions, but devoid of a nucleic acid described herein (e.g., a heterologous promoter operably linked to a nucleic acid encoding an ILP1 protein). In one aspect, an increase in seed oil yield refers to increased harvestable seed oil. The quantity of seed oil in accordance with the methods herein may be calculated on a per plant, per seed, or per 10 seed basis or in relation to a specific area, seed oil obtained per acre/square meter/or the like. Mass of oil per plant may also be used as a unit of measure and may be calculated from total seed weight. In some aspects, seed oil obtained from a population of plants may be calculated in relation to a hectare of land, i.e., seed oil produced per hectare.

Seed oil content may be measured by conventional techniques. For example, Nuclear Magnetic Resonance Spectrometry (NMR) is used for determining oil, protein, and moisture content in seeds of various oilseed crops. The instrument is calibrated for several oil crops including soybean, camelina, canola, meadowfoam, and euphorbia, and can be calibrated for other species as needed. Tests may be performed in whole seeds or meals. The oil content test estimates the amount of oil that may be extracted from seeds using an industrial extraction method. Direct pressing extracts 90-92%, whereas solvent extractions remove 97-99% of oils contained in seeds. The NMR technique measures the resonance energy absorbed by hydrogen atoms in the sample. Usually oil contents are expressed based on a specific moisture basis (e.g., 8.5%, 10%, etc.). Using the NMR in measuring oil content is referenced by ISO 10565:1998 Oilseeds. The typical oil content in seeds of different feedstock may vary, e.g., oil content of castor seeds, soybean, sunflower seeds, and rapeseed is 40-55%, 15-20%, 25-35%, and 38-46%, respectively.

“Seed protein” refers to the protein extracted or extractable from seeds of an oilseed plant. Increased “seed protein yield” means that the amount or quantity of seed protein obtained from the respective population of plants is increased by a measurable amount over the amount or quantity of seed protein obtained from the same population of plants produced under the same conditions, but devoid of a nucleic acid described herein (e.g., a heterologous promoter operably linked to a nucleic acid encoding an ILP1 protein). In one aspect, an increase in seed protein yield refers to increased harvestable seed protein.

“Seed starch” refers to the starch extracted or extractable from seeds of an oilseed plant. Increased “seed starch yield” means that the amount or quantity of seed starch obtained from the respective population of plants is increased by a measurable amount over the amount or quantity of seed starch obtained from the same population of plants produced under the same conditions, but devoid of a nucleic acid described herein (e.g., a heterologous promoter operably linked to a nucleic acid encoding an ILP1 protein). In one aspect, an increase in seed starch yield refers to increased harvestable seed starch.

An improvement in fitness of a plant may refer to increasing the production of a plant, for example improving the yield of the plant, improving the vigor of the plant, or the quality of the product harvested from the plant.

“Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Plants are more susceptible to drought during flowering and seed development (the reproductive stages), as plant's resources are deviated to support root growth. Accordingly an improvement in drought tolerance refers to the ability of a plant to grow, develop, or yield normally, or grow, develop, or yield faster or better than normal when subjected to reduced amounts of available/applied water and/or under conditions of acute or chronic drought; ability of a plant to grow, develop, or yield normally when subjected to reduced amounts of available/applied water (water input) or under conditions of water deficit stress or under conditions of acute or chronic drought.

Increased disease and/or pest resistance refers to an increased resistance or tolerance to diseases such as bacterial, viral or fungal diseases and/or pests such as insects, arachnids, nematodes, mites, slugs, and/or snails. An improvement in disease and/or pest resistance refers to the ability of a plant to grow, develop, or yield normally, or grow, develop, or yield faster or better than normal when subjected to a disease and/or pest.

The “harvestable yield” of a plant may depend on the specific plant/crop of interest as well as its intended application (such as food production, feed production, processed food production, biofuel, biogas or alcohol production, or the like) of interest in each particular case. Thus, in one aspect, yield is calculated as harvest index (expressed as a ratio of the weight of the respective harvestable parts (e.g., seed, seed oil, seed protein, or seed starch) divided by the total biomass), harvestable parts weight per area (acre, square meter, or the like), and the like.

An improvement in one or more of the agronomic traits of a modified plant described herein may be an increase of at least about 0.5%, about 1%, about 2%, about 3%, about 4%, about 5%, about 10%, about 20%, about 30%, about 40%, about 50%, about 60%, about 70%, about 80%, about 90%, about 100%, or more than about 100% as compared to the same variety or species of plant grown under the same conditions, but devoid of a nucleic acid described herein (e.g., a heterologous promoter operably linked to a nucleic acid encoding an ILP1 protein).

In another aspect, a method of improving one or more agronomic traits of a plant is provided, which comprises killing or inhibiting the growth of the shoot apical meristem of a modified plant described herein, e.g., a modified plant comprising a heterologous promoter operably linked to a nucleic acid encoding an IPL1 polypeptide, and optionally comprising a second heterologous promoter operably linked to a nucleic acid encoding an G6DP polypeptide and/or invertase polypeptide. In some aspects, the method further includes the step of harvesting a commercially relevant part of the modified plant. The term “commercially relevant part of a plant” refers to a plant part (e.g., leaf, root, tuber, seed, nut, or fruit), or one or more natural plant products such as oil, protein, carbohydrates (e.g., sugar or starch), fiber, terpenes, or more. In some aspects, the method further includes the step of harvesting an oil, protein, or a combination thereof from the modified plant or plant part thereof.

As used herein, the term “meristem” means a region of cells capable of division and growth in plants. Meristematic cells are typically small and nearly spherical. They have a dense cytoplasm and relatively few small vacuoles. Some of these meristematic cells maintain the meristem as a continuing source of new cells and may undergo cell division (mitosis) many times before differentiating into specific cells required for that region of the plant body. An “apical meristem” refers the meristem at the shoot or root tips of the plant. The apical meristems give rise to the primary plant body and are responsible for the extension of the roots and shoots. The “shoot apical meristem” is a center of potentially indefinite growth and development producing the leaves as well as a bud in the axil of most leaves that has the potential to grow out as a branch. The shoot apical meristem generates all the aerial organs including the floral meristems. The plant hormone auxin is produced in the shoot apical meristem. Among the many roles of auxin in plant development, it inhibits the production of lateral branches.

In some aspects, a plant herein includes, but is not limited to, cereals, for example wheat, rye, barley, triticale, oats or rice; beet, for example sugar beet or fodder beet; fruits, such as pomes, stone fruits or soft fruits, for example apples, pears, plums, peaches, almonds, cherries, strawberries, raspberries, blackberries or gooseberries; leguminous plants, such as lentils, peas, alfalfa or soybeans; cucurbits, such as squashes, cucumber or melons; fiber plants, such as cotton, flax, hemp or jute; citrus fruit, such as oranges, lemons, grapefruits or mandarins; vegetables, such as broccoli, spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, for example conifers; and on the plant propagation material, such as seeds, and the crop material of these plants. In some aspects, the plant is a leguminous plant, such as lentil, pea, peanut, chickpea, kidney bean, lupine, alfalfa or soybean. In other aspects, the plant is soybean, corn, canola, rice, potato, camelina, or sunflower. A plant of the invention does not include plants in the genus Arabidopsis.

In some aspects, the modified plant is a modified oilseed plant. An “oilseed plant” is a plant that is grown or cultivated for production of oils from the seeds of the plant, e.g., edible oils, edible oils used as food supplements or nutraceutical, multipurpose oils, inedible oils as biofuel, drying oils, and/or oils used in industrial applications and commercial products. In some aspects, the oilseed plant is a plant grown or cultivated as a row crop. Edible oils include the following, which are also used as fuel oils, corn oil, cottonseed oil, peanut oil, rapeseed oil, safflower oil, sesame oil, soybean oil, and sunflower oil. Other edible oils include Amaranth oil from the seeds of grain amaranth species, including Amaranthus cruentus and Amaranthus hypochondriacus; false flax oil from seeds of Camelina sativa; hemp oil; meadowfoam seed oil; mustard oil; Quinoa oil; ramtil oil pressed from the seeds of the one of several species of genus Guizotia abyssinica (Niger pea); rice bran oil; thistle oil pressed from the seeds of Silybum marianum; tobacco seed oil from the seeds of Nicotiana tabacum and other Nicotiana species; tomato seed oil; and wheat germ oil. Oils used as food supplements or nutraceuticals include black seed oil pressed from Nigella sativa seeds, borage seed oil from the seeds of Borago officinalis, evening primrose oil from the seeds of Oenothera biennis, and flaxseed oil (called linseed oil when used as a drying oil) from the seeds of Linum usitatissimum. In some aspects, modified oilseed plants used in the methods described herein are soybean, rapeseed, canola, camelina, sorghum, safflower, sunflower, corn, sesame, cotton, cress, peanut, castor, flax, mustard, quinoa, chia, cuphea, and amaranth plants.

Oils used primarily for human consumption, but which have been considered for use as biofuels, i.e., multipurpose oils, include castor oil; colza oil from Brassica rapa var. oleifera (oilseed turnip); corn oil; cottonseed oil; false flax oil from Camelina sativa; hemp oil; mustard oil; peanut oil; radish oil; rapeseed oil; rice bran oil; safflower oil; soybean oil; and sunflower oil.

Vegetable oils that dry to a hard finish at normal room temperature are referred to as “drying oils” and are used as the basis of oil paints and in other paint and wood finishing applications. Such oils include sunflower and safflower oil; linseed (flaxseed) oil; and Vernonia oil produced from the seeds of the Vernonia galamensis.

Other plant oils of importance that are either inedible, or which are not commonly ingested as edible oils, can be used for a wide variety of other purposes including, for example, insecticides, perfumes, various industrial applications, sources of triglycerides and fatty acids, medicinal and cosmetic uses, etc. These include, for example, bladderpod oil pressed from the seeds of Lesquerella fendleri; carrot seed oil pressed from carrot seeds; castor oil; crambe oil extracted from seeds of Crambe abyssinica; and honesty oil from seeds of Lunaria annua.

Soybean (Glycine max) oil is a co-product with soybean meal. To manufacture biodiesel from soybean oil, harvest beans are crushed, and the meal and oil separated.

Rapeseed (Brassica napus subsp. napus) has a deep root system that allows the plants to scavenge for water and nutrients. The rapeseed plant is allowed to grow and mature normally until bolting, when the main flower stem begins to develop. When the main flower stem is still young, the apical meristem is removed which forces the plant to branch from remaining nodes. Low erucic acid varieties of canola also may be used.

Mustard plants, including black mustard (Brassica nigra), brown mustard (B. juncea), and white mustard (Sinapis alba), are similar to canola and rapeseed. Mustard is an excellent cover crop with excellent potential as a biodiesel feedstock. Although mustard produces less oil than rapeseed or canola, it is more drought tolerant and grows well on marginal soils. The mustard plant is allowed to grow and mature normally until bolting, when the main flower stem begins to develop. When the main flower stem is still young, it may be removed thereby removing apical dominance and forcing the plant to branch from remaining nodes.

Camelina (Camelina sativa) is an oil seed crop that is a relative of mustard, rapeseed, and canola. In some locations, camelina can be grown at a lower cost than rapeseed or canola because it requires less fertilizer and pesticides. Camelina has many favorable attributes for biodiesel production including high plant yield, high oil content in the seed, high net energy ratio, and relatively low production cost. Camelina has many favorable attributes for fuel production including high plant yield, high oil content in the seed, high net energy ratio, and comparatively low production cost. Camelina has a short life cycle of about 85-100 days from seeding to maturity making it an attractive crop for oil seed production.

Safflower (Carthamus tinctorius) is a highly branched, thistle-like annual in the family Asteraceae in which vegetable oil is extracted from seeds. Safflower originates from a leafy rosette from which a branched central stem emerges. The main shoot reaches heights of 12-59 inches. Lateral branches develop once the main stem is about 7.9-15.7 inches high. These lateral branches can then branch again to produce secondary and tertiary branches. The chosen variety as well as growing conditions influence the extent of branching. The shoot apical meristem may be removed anywhere from growth state 1 to growth stage 6 or higher on safflower during its growth period prior to flowering to force branching and the production of additional fruits.

Flax (Linum usitatissimum) is an herbaceous annual in the family Linaceae that is cultivated for its fiber, which produces linen, and for its seed, called flaxseed or linseed, from which linseed oil is produced. It has the highest alpha-linolenic acid levels of any other vegetable oils. Flax seeds are generally made up of approximately 20% protein and 41% oil concentrations. When flax germinates, the first parts to emerge from the ground are the cotyledons. These expand and from the middle of them, the apical meristem grows upwards, putting out new leaves. Three pairs of opposite, true leaves are the next to grow. The stem then continues to elongate, putting out small leaves that spiral up the main stem, until branching begins. All of this is part of the vegetative stage of the plant, which takes about 45-60 days. The flowering period (the reproductive stage) takes 15-25 days. The flowering period is when branching begins near the top of the plant and flowers emerge. Flowers remain open for approximately a day, and then close up while flax seeds mature inside the pod. Seeds mature and the plants dry down, which takes another 30-40 days. Removal of the shoot apical meristem may occur just above the first set of true leaves, the second set of true leaves, or the third set of true leaves, early in the life cycle of the plant. This is achieved, e.g., by clipping or treating the shoot apical meristem with a chemical composition that kills sufficient cells, or inhibits the growth of sufficient cells, to stop or inhibit the growth of the apical meristem. This results in a few large branches low on the plant. How many true leaves are remaining determines how many potential branches can be formed.

Sesame (Sesamum indicum) is a perennial plant and is one of the oldest domesticated oilseed crops. It tolerates drought conditions well and has high oil concentrations in the seeds, generally about 60%. Sesame grows 20-39 inches tall, with opposite leaves 2 to 6 inches long, lanceolate in shape, 2 inches broad at the base of the plant, narrowing to just 0.4 inches broad on the flowering stem. Fruit capsules containing 4-12 seeds are produced from the base of the leaf nodes. The shoot apical meristem may be removed anywhere from growth stage 1 to the growth stage 6 or higher on sesame during its growth period prior to flowering to force branching and the production of additional fruits.

Sunflower is a large annual in the genus Helianthus. Wild H. annuus is widely branched with many flower heads. The domestic sunflower often possesses only a single large inflorescence on an unbranched stem. Oil is extracted from sunflower seeds. After emergence of a sunflower plant from seed, the cotyledons expand with opposite nodes, followed by the first set of true leaves, also from opposite nodes. The stem elongates adding leaves alternating up the plant until the flower(s) develop. After pollination, each of the flowers produce seeds, which then develop, mature, and dry. Removal of the shoot apical meristem may be achieved by clipping or applying a composition to the main stem just above the cotyledonary nodes and below the next node of opposite leaves. Shoot apical meristem removal at this time leaves 2 remaining nodes from which the plant can grow branches. Shoot apical meristem removal may also take place before the next higher leaves are fully formed or after they have formed. Shoot apical meristem removal may also take place above the set of opposite nodes (which are just above the cotyledonary nodes). This may also occur before the leaves of the next node fully form or after they form. Shoot apical meristem removal at this stage leaves 4 potential nodes from which branches can form. Most commonly, for both of these heights, 2 branches form from opposite nodes (either cotyledonary or the set higher) and both act like main stems. Alternatively, the shoot apical meristem of a sunflower plant may be removed above one of the next approximately 5 leaves that are alternately located up the main stem. This can result in 1-3 branches forming from the highest remaining nodes.

In some aspects, the growth of a shoot apical meristem of a modified plant herein may be inhibited or the shoot apical meristem may be killed by clipping. The term “clipping” means removal or inhibition of the growth of the shoot apical meristem of a plant by any means of mechanical trimming. Mechanical trimming is accomplished by mowing, pruning by hand, pinching, plucking, or any other method of severing the shoot apical meristem in whole, or in part, from the plant. In some aspects, clipping is carried out when the modified plant has reached at least a growth stage of V3. In some aspect, a modified plant is clipped above a first vegetative node.

In some aspects, the growth of a shoot apical meristem of a modified plant herein may be inhibited or the shoot apical meristem may be killed with a chemical inhibitor. Chemical treatment is accomplished using any chemical composition that inhibits the growth or kills a significant number of cells in the shoot apical meristem, without significantly damaging growth of other parts of the plant. In some aspects, the chemical inhibitor is a composition comprising one or more fatty acid esters (e.g., methyl decanoate, and/or methyl nonanoate), fatty alcohols (e.g. 1-decanol), and optionally one or more surfactants; or a composition comprising 4% of the methyl ester of the C6 fatty acid, about 56% of the methyl ester of the C8 fatty acid, about 38% of the methyl ester of the C10 fatty acid, and about 2% of the methyl ester of the C12 fatty acid, based on total fatty acid of the composition. See, e.g., U.S. patent application Ser. No. 18/405,062, filed Jan. 5, 2024 and U.S. patent application Ser. No. 18/650,973, filed Apr. 30, 2025, incorporated herein by reference in their entireties.

In accordance with the methods herein, an effective amount of a chemical inhibitor may be applied to the shoot apical meristem of a population of oilseed plants. As used herein, the term “applying,” “applied,” “application,” “contact,” “contacting” or variations thereof, with reference to the composition means that the shoot apical meristem is contacted with the composition using any suitable means, e.g., sprayed, drenched, or misted. Various applicators may be used to apply the composition to the shoot apical meristem including, e.g., a hand-held aspirator-type sprayer or other commercial sprayer. Examples of commercial sprayers include the Hagie STS12, STS16 and STS20 models capable of carrying 1200, 1600 and 2000 gallons, respectively, of spray product. Such commercial sprayers may need to be modified with spray arms to target the apical meristems from the upper side of the plants. In some aspects, the spray includes a pressurized nozzle, e.g., a nozzle having a psi of about 40.

To effect overcompensation or an increase in the level of endopolyploidy leading to an increase in one or more agronomic traits, some aspects provide for the killing or inhibition of the growth of the shoot apical meristem at a specified growth stage. The growth stage at which the shoot apical meristem is killed or inhibited may be dependent upon the oilseed plant species/variety, geographical location, desired increase in the one or more agronomic traits, as well as other factors including environmental conditions such as soil, temperature, light, disease pressure, water availability, drought, nutrient availability, which are known to those of skill in the art to influence plant growth.

Depending, in part, on genetics, a plant selection, plant variety, inbred plant, or hybrid plant may not express overcompensation when cells in the apical meristem are killed or their growth inhibited. Thus, plants may be screened to identify those capable of expressing overcompensation, e.g., by treatment of the shoot apical meristem with a composition described herein or clipping and measuring, e.g., seed yield to determine whether there is an increase in seed yield in the treated plant as compared to a plant not treated with the composition or clipped. Furthermore, the optimal time in the plant growth cycle for removal, or inhibition of growth, of the shoot apical meristem may be determined by treatment of the shoot apical meristem with a composition described herein at different stages of growth and determining the stage in which there is an increase in, e.g., seed yield as compared to a plant not treated with the composition.

Alternatively, or in addition to, screening of plants and/or growth stage may be conducted at the molecular level. Increasing chromosome number through endoreduplication and therefore gene copy number provides a means of increasing expression of vital genes or genetic pathways that promote rapid regrowth rates following removal, or inhibition of growth, of the apical meristem. Endoreduplication leads to increased gene copy number and therefore increased gene expression. Accordingly, gene expression of, e.g., G6PD1, may be measured to assess appropriate plants and/or growth stages for use in this invention.

It is understood that a skilled person will be able to determine/identify the different growth stages of a particular crop concerned based upon conventional industry scales. Particular guidance for determining the growth stage of soybean (Glycine max L.) is provided by Soybean Growth Stages, published by the University of Illinois in 1999 (see, e.g., Najafikhan-Behbin, et al. (2019) Appl. Ecol. Environ. Res. 17(2):2911-2929) and McWilliam, et al. (1999) Soybean Growth and Management Quick Guide, A-1174, North Dakota State University, Frago, ND. Guidance for determining the growth stages of camelina (Camelina sativa) may be found in Martinelli & Glasso (2011) Annals Appl. Biol. 158:87-94. Guidance for determining the growth stages of peanut (Arachis hypogaea L.), cotton (Gossypium hirsutum L.), corn (Zea mays L.), canola (Brassica napus L.), and sunflower (Helianthus annuus L.) may be found in Meier (2001) Biologische Bundesanstalt, Bundessortenamt and CHemical (BBCH) Monograph. The BBCH scale uses a decimal-E code system, which is divided into principal and secondary growth stages, and is based on the cereal code system (Zadoks scale) developed by Zadoks et al. (1974) Weed Res. 14:415-421. Guidance for determining the growth stages of sorghum may be found at the Sorghum Checkoff Industry Organization (sorghumcheckoff.com/our-farmers/grain-production/growth-and-development/).

By way of illustration, removal, killing of a significant number of cells, or inhibition to growth of the shoot apical meristem of soybean (Glycine max L.) may occur between (i) growth stage 1 and growth stage 2, (ii) growth stage 2 and growth stage 3, (iii) growth stage 3 and growth stage 4, (iv) growth stage 4 and growth stage 5, or (v) growth stage 5 and growth stage 6. For soybeans, VE is the stage at which cotyledons emerge and supply energy for the plant for 7 to 10 days. VC is the stage soon after the cotyledons are fully exposed. At VC unifoliate leaves emerge at the second node and begin generating energy through photosynthesis. Growth state 1 or the V1 stage is when the first trifoliate leaflets form. The V2 stage (growth stage 2) is when the second trifoliate leaf is established. At V2 root nodules begin to develop. Nitrogen fixation by the plant begins to occur when the plants reach 6 to 8 inches in height. V3 (growth stage 3) is the stage when the third trifoliate leaf emerges. V4 (growth stage 4) is when the fourth trifoliate leaf emerges. V5 (growth stage 5) is when the fifth trifoliate leaf emerges. The V6 stage (growth stage 6) is when plants develop new growth stages about every 3 days depending on the environment. At this stage lateral roots should overlap in 30-inch rows or less. At R1 flowering begins on the third to sixth node and continues up and down the main stem. Flowing eventually moves to the branches. Nodes on the main stem usually have at least one flower. Vertical roots as well as secondary roots and root hairs continue to grow rapidly until R4 and R5. At stage R2 an open flower develops at one of the top two modes of the main stem. The plant has accumulated about 25% of its total dry weight and nutrients and about 50% of its mature height. Nitrogen fixation by root nodules increases rapidly at this stage. At stage R3 a pod on at least one of the upper four nodes is at least 3/16-inch long or longer. At stage R4 the pods are growing rapidly, and seeds are developing. At stage R5 at least one ⅛-inch-long seed is present in a pod at one of the four upper-most nodes. About half of the nutrients required for seed filling come from the plant's vegetative parts and about half from N fixation and nutrient uptake by the roots. Nitrogen fixation peaks at this stage. Stage R6 is known as the “green bean” stage because it marks the beginning of the full seed stage. At stage R7 at least one normal pod on the main stem reaches its brown or tan mature color. Seed dry matter begins to peak. Seeds and pods begin to lose green color. At stage R8 at least 95% of the pods on a plant have reached their mature color and the plant is fully mature. Typically, about 5 to 10 days of good drying weather after stage R8 is needed to obtain a harvest seed moisture content of less than 15%.

For corn (Zea mays), a composition as described herein may be applied to the shoot apical meristem of the corn between (i) growth stages 9 and 10, (ii) growth stages 10 and 11, (iii) growth stages 11 and 12, (iv) growth stages 12 and 13, (v) growth stages 13 and 14, (vi) growth stages 14 and 15, (vii) growth stages 15 and 16, (viii) growth stages 16 and 17, (ix) growth stages 17 and 18, (x) growth stages 18 and 19, or (xi) growth stages 19 and 30. Similar to dicots, growth stage of a monocot such as corn is when the first round-tipped leaf on first collar appears, and nodal roots elongate. By growth stage 2, the monocot may be 2 to 4 inches tall and rely on energy in the seed. Growth stage 3 begins 2 to 4 weeks after VE (emergence), and the plant switches from kernel reserves to photosynthesis and nodal roots begin to take over. Notably, in corn, a plant with 3 collars is considered growth stage 3, however, there may be 5 to 6 leaves showing on the plant. At the growth stage 4 stage, the fourth leaf collar is visible. At growth state 5-6, leaf collars are visible, the growing point is above the soil surface, the critical period of nitrogen uptake begins, and kernel row numbers are determined. See the Biologische Bundesanstalt, Bundessortenamt and Chemical (BBC-1) scale for corn.

In some aspects, the oilseed plant is canola and the composition is applied to the shoot apical meristem of the canola between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 20. See the BBCH scale for canola.

In some aspects, the oilseed plant is camelina and the composition is applied to the shoot apical meristem of the camelina between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 21. See the BBCH scale for camelina.

In some aspects, the oilseed plant is sunflower and the composition is applied to the shoot apical meristem of the sunflower between (i) growth stages 10 and 12, (ii) growth stages 12 and 14, (iii) growth stages 14 and 16, (iv) growth stages 16 and 18, or (v) growth stages 19 and 30. See the BBCH scale for sunflower. See the BBCH scale for sun flower.

In some aspects, the oilseed plant is cotton and the composition is applied to the shoot apical meristem of the cotton between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 21. See the BBCH scale for cotton.

In some aspects, the oilseed plant is peanut and the composition is applied to the shoot apical meristem of the peanut between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 21. See the BBCH scale for peanut.

In some aspects, the oilseed plant is sorghum and the composition is applied to the shoot apical meristem of the sorghum between (i) growth stages 0 and 1, (ii) growth stages 1 and 2, (iii) growth stages 2 and 3, (iv) growth stages 3 and 4, (v) growth stages 4 and 5, or (vi) growth stages 5 and 6. See the Sorghum Checkoff Industry Organization developmental stages of the sorghum plant.

Ideally, removal of the shoot apical meristem results in minimal or no removal of adjacent growth stage. For example, a method herein provides for minimal or no removal of adjacent growth stage 1 if removing the apical meristem between growth stage 1 and growth stage 2, or minimal or no removal of adjacent growth stage 2 tissue if removing the apical meristem between growth stage 2 and growth stage 3, or minimal or no removal of adjacent growth stage 3 tissue if removing the apical meristem between growth stage 3 and growth stage 4, or minimal or no removal of adjacent growth stage 4 tissue if removing the apical meristem between growth stage 4 and growth stage 5, or minimal or no removal of adjacent growth stage 5 tissue if removing the apical meristem between growth stage 5 and growth stage 6, etc.

Given that growth stages may overlap, a crop of plants is in a particular growth stage when 50% or more of the plants of the crop are in or beyond that stage. Moreover, if senescence of the lower leaves has occurred, leaf scars (excluding those where the cotyledons were attached) are counted to determine the proper stage. Removal or inhibition of the growth of the apical meristem may occur within the 3-to-10-day window between stages, e.g., between stages 1 and 2, stages 2 and 3, stages 3 and 4, etc. In addition, depending on the plant variety, geographic region, or agronomic conditions, the apical meristems of the plants may be removed early or later in plant growth.

The following examples are provided for illustrative purposes only and not intended to limit the scope of the claims.

### Example 1: Materials and Methods

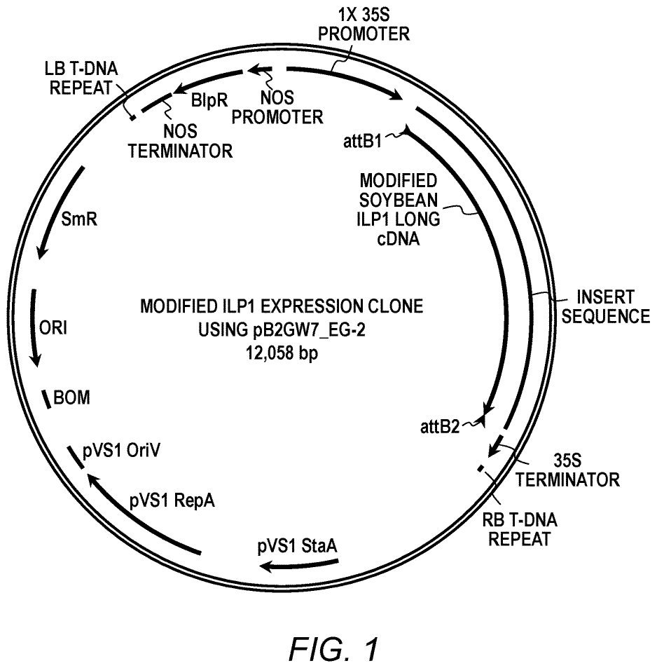

Plasmid vector pB2GW7 was used to clone ILP1 and associated genes for transformation into soybean (Williams 82) using disarmed Agrobacterium tumefaciens. The vector included a single copy of the ILP1 coding sequence encoding soybean ILP1 (SEQ ID NO:2) operably linked to a CaMV (Cauliflower mosaic virus) 35S promoter and CaMV 35S terminator. The vector was designed to increase reproductive success of transformed and clipped soybean over a transformed control soybean plant. Alternatively stated, the Williams 82 soybean cultivar was changed from an undercompensator to an overcompensator by increasing ILP1 expression, thereby increasing ploidy and consequently increasing seed production. In addition to ILP1, the vector included a Bialaphos (Bar) resistance gene operably linked to an A. tumefaciens NOS promoter and NOS terminator to facilitate screening for successful transformation events. In addition, the vector include a bacterial streptomycin resistance gene from Streptomyces griseus (FIG. 1).

Soybean was transformed by conventional Agrobacterium-mediated transformation. Successful transgenic events were each verified with herbicide resistance using Liberty herbicide. All modified soybeans were grown in a greenhouse. The first experiment (Experiment 1) included two modified lines (lines 1 and 10) and the wild-type cultivar, Williams 82. The second experiment (Experiment 2) included three additional modified plants (lines 31, 33, and 40), seeds harvested from a single plant from each of Lines 1 (plant 1-32) and 10 (plant 10-9) in which each showed a positive response to clipping, as well as the wild-type cultivar, Williams 82. Additional lighting was provided by 1000-watt halide bulbs set on a 12-hour cycle. All plants were grown in 2.5 L pots, filled with a soil mix of 1:1:1 soil, perlite, and torpedo sand. Pots were initially seeded with 1-4 seeds, depending on the number of seeds available, to make sure each pot had at least one successful germination. Once the plants had emerged and reached the unifoliate stage, pots were thinned so that all the plants were as healthy and homogenous as possible. Watering was carried out frequently to ensure the pots stayed wet and once plants reached the V3 stage, the plants were fertilized weekly with a 24-8-16 fertilizer (Scotts Miracle-Gro Product, Inc, Marysville, OH). In each experiment, plants were randomly assigned as either unclipped controls or were experimentally clipped. Clipping was carried out when plants reached the V2 stage, meaning that the trifoliate at the second node was fully expanded. Plants were clipped above the first vegetative node when the plant had a fully expanded leaf at the second vegetative node.

When most unclipped plants reached V7 (the seventh trifoliate was fully expanded), half of one leaflet from the second-highest trifoliate was painted with Liberty herbicide, at a 3:100 dilution with water to determine which plants carried the transgene. Herbicide-applied leaves were monitored for the next two weeks and were scored on a scale from 1-10 for damage. Plants that scored a 1 had no visible changes, plants that scored a 2-5 showed increasing amounts of damage including yellow and brown spots and leaf curling but there is no other damage. Much of the damage in the plants that scored 2-5 occurred at the tip of the leaf where the diluted herbicide had pooled before drying, resulting in more active ingredient at those spots, in turn explaining the increased damage. Finally, plants that scored 6-10 had much higher amounts of damage, usually resulting in necrosis over the entire half of the leaf that was treated with the diluted herbicide and were therefore classified as sensitive to the herbicide. Two more weeks after the initial scoring, all the herbicide-treated leaves were re-scored for verification. Most of the plants that were in the sensitive category showed complete leaf death on the half of the leaf where the herbicide was applied, scoring a “10” and verifying sensitivity. Plants with a score of 6 or higher were classified as sensitive to the herbicide, likely meaning they did not have two copies of a fully functioning transgene and that the transgene was likely lost via recombination.

To assess the level of endoreduplication, leaf samples were taken from the modified lines 33, 40, and the wild-type line (Williams 82) at the beginning of pod fill during the second experiment, as this timing has previously been determined to correspond to the highest levels of endoreduplication. Modified lines 33 and 40 were selected as they appeared to show the highest potential for enhanced fitness following the clipping treatment. These were analyzed for the level of endoreduplication.

Once plants reached R8 and their full pods turned brown, no water was added to the pots, thus allowing the plants to dry down. After harvesting by hand, architectural data such as branch and node number, as well as yield-relevant data (pod number, seed number, and seed weight) were collected. Seeds were then analyzed for protein and oil concentration (adjusted to 13% moisture content) by a near-infrared (NIR) reflectance diode array feed analyzer (Perten, Spring Field, IL). After the first planting, representative samples of plants from lines 1, 10, and Williams 82 were selected for nodule counts, as well as all of the plants from lines 33, 40, and Williams 82 in the second planting. Pots were submerged in a water bath and carefully massaged until all the soil was washed from the roots, allowing nodules to be counted. Clipped plants were compared to unclipped plants using one-tailed paired t-tests, as well as independent t-tests followed by a multiple range test.

### Example 2: Manipulation of Endoreduplication in Soybean and its Effects on Overcompensation

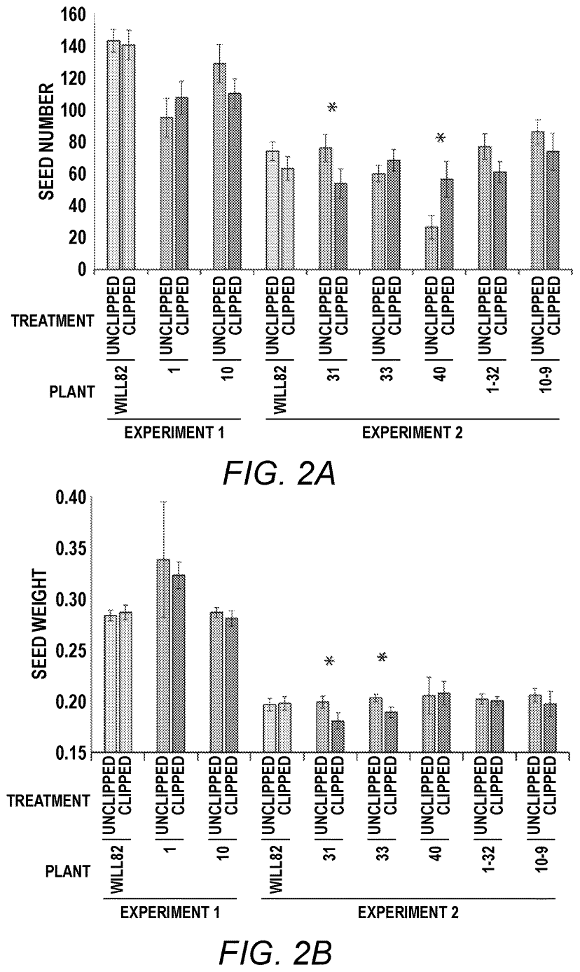

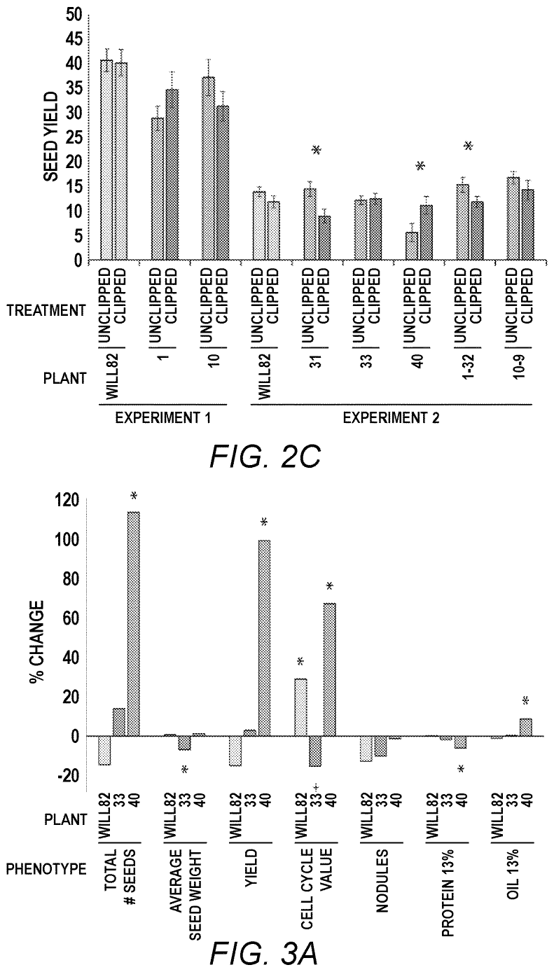

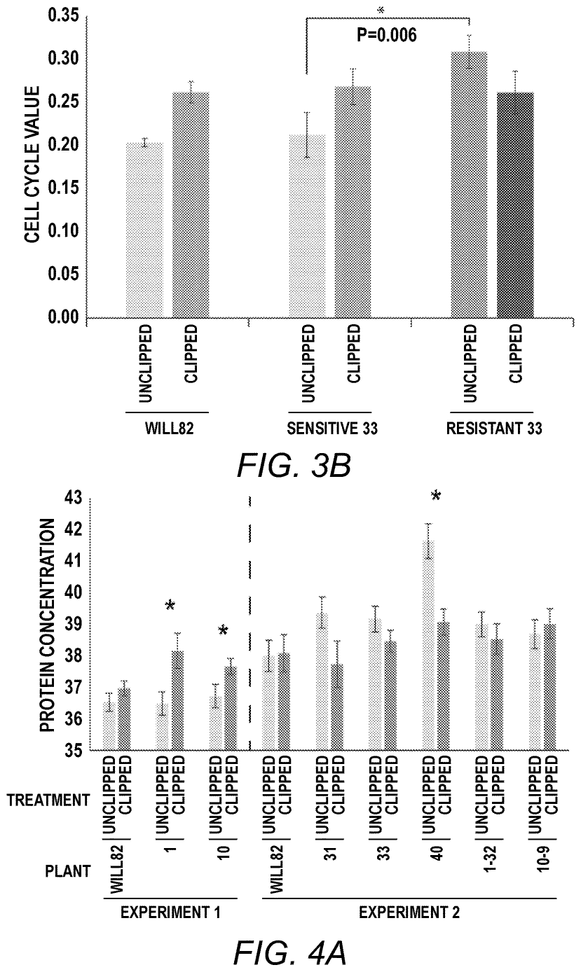

Five successful modified plants were tested over the two experiments, each designated by modified plant number: 1, 10, 31, 33, and 40. When comparing herbicide-resistant (and therefore transgenic) clipped plants to their unclipped controls, modified plant line 40 had a significant increase in seed production (p=0.036; FIG. 2A). Even though plant line 40 did not have a significant change in seed weight (p=0.455; FIG. 2B), seed yield (seed number×seed weight) of this line still increased significantly (p=0.037; FIG. 2B). Line 40 also had a corresponding increase in the level of endopolyploidy (i.e., cell cycle value) over the unclipped control (p=0.004; FIG. 3A). Plants from line 31 showed a significant decrease in seed number, seed weight, and seed yield (p=0.043, 0.036, and 0.007, respectively; FIGS. 2A-2C). Modified line 1 plants that were grown in the second experiment (denoted “1-32,” as this was plant 32, which was the largest of the clipped plants) had a marginally significant decrease in seed number, no change in seed weight, and a significant decrease in yield (p=0.074, 0.411, and 0.043; FIGS. 2A-2C). Line 33 had an insignificant increase in seed number and a significant decrease in seed size, resulting in no change in yield (p=0.163, 0.018, and 0.405, respectively; FIGS. 2A-2C). Line 33 had a marginally significant decrease in cell cycle value (p=0.072; FIGS. 3A-3B). All other modified lines had insignificant changes in seed number, seed weight, and total yield (p>0.05; FIGS. 2A-2C). In both growing sessions, the wild-type Williams 82 had an insignificant decrease in seed number, weight, and yield (P>0.05). In the first experiment, no measurements of endopolyploidy were taken, but in the second experiment, Williams 82 had a significant increase in cell cycle value (p=0.003; FIG. 3A).

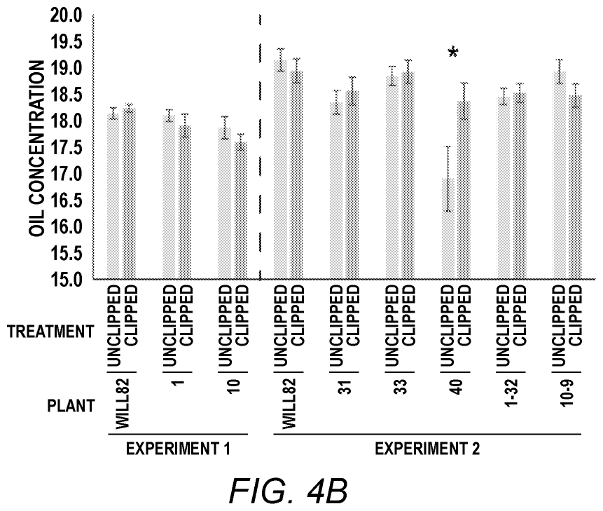

When comparing the resistant clipped plants to their unclipped controls, none of the lines had significant changes in nodule number, even though there were significant changes in some of the lines for yield components. Protein and oil concentrations changed after clipping in some lines, as compared to the unclipped controls (FIGS. 4A-4B). For example, clipped plants from lines 1 and 10 showed significant increases over their unclipped controls (p=0.013 and 0.026) while line 40 showed a significant decrease in protein concentration (p=0.005). Only line 40 showed a significant increase in oil concentration over the unclipped control (p=0.04). All other lines showed insignificant changes in either protein or oil concentrations after clipping (p>0.05), as compared to the unclipped controls.

What is claimed is:

1. A modified plant or plant part thereof comprising a nucleic acid encoding an Increased Level of Polyploidy 1 (ILP1) protein, wherein the nucleic acid comprises: (a) a protein-coding sequence encoding the ILP1 protein, and (b) one or more regulatory elements operably linked to the protein-coding sequence.

2. The modified plant or plant part thereof of claim 1, wherein (a) and/or (b) are from the same plant species as the modified plant.

3. The modified plant or plant part thereof of claim 1, wherein (a) and/or (b) are from a different plant species as the modified plant.

4. The modified plant or plant part thereof of claim 1, wherein (a) and/or (b) are from a dicotyledonous plant.

5. The modified plant or plant part thereof of claim 1, wherein at least (a) and/or (b) are from a monocotyledonous plant.

6. The modified plant or plant part thereof of claim 1, wherein (a) and (b) comprise an ILP1 gene.

7. The modified plant or plant part thereof of claim 1, wherein the one or more regulatory elements operably linked to the protein-coding sequence comprise a heterologous promoter.

8. The modified plant or plant part thereof of claim 7, wherein the heterologous promoter is selected from the group consisting of a cauliflower mosaic virus 35S promoter, cauliflower mosaic virus 19S promoter, nos promoter, Adh1 promoter, sucrose synthase promoter, α-tubulin promoter, ubiquitin promoter, actin promoter, cab promoter, PEPCase promoter, R gene complex promoter, poplar xylem-specific secondary cell wall specific cellulose synthase 8 promoter, phaseolin promoter, and Arabidopsis Ubiquitin 10 promoter.

9. The modified plant or plant part thereof of claim 1, wherein the modified plant is an oilseed plant selected from the group consisting of soybean, rapeseed, canola, camelina, sorghum, safflower, sunflower, corn, sesame, cotton, cress, peanut, castor, flax, mustard, quinoa, chia, cuphea, and amaranth plants.

10. A method for improving one or more agronomic traits of a plant or plant part thereof comprising killing or inhibiting the growth of a sufficient number of cells in the shoot apical meristem of the modified plant of claim 1, thereby improving one or more agronomic traits of the plant or plant part thereof.

11. The method of claim 10, wherein the one or more agronomic traits comprise increased fitness, seed yield, drought tolerance, pest/pathogen resistance, disease resistance, seed pod yield, biomass yield, seed protein yield, seed oil yield, or seed starch yield.

12. The method of claim 11, wherein the modified plant is an oilseed plant selected from the group consisting of soybean, rapeseed, canola, camelina, sorghum, safflower, sunflower, corn, sesame, cotton, cress, peanut, castor, flax, mustard, quinoa, chia, cuphea, and amaranth plants.

13. The method of claim 10, wherein the shoot apical meristem is killed by clipping.

14. The method of claim 13, wherein the clipping is carried out when the transgenic oilseed plant has reached a vegetative growth stage of 1 to 6.

15. The method of claim 12, wherein the modified plant is clipped above a first vegetative node.

16. The method of claim 11, wherein the shoot apical meristem is inhibited with a chemical inhibitor.

17. The method of claim 10, further comprising harvesting an oil, protein, or a combination thereof from the modified plant or plant part thereof.

18. The method of claim 17, wherein the plant part is a fruit or seed.
